SEQUENCE LISTING (70 sequences)

SEQ ID NO: 1 - protein, 10 aa, Artificial Sequence
  Description of Artificial Sequence Synthetic peptide
GGGGSGGGGS

SEQ ID NO: 2 - protein, 25 aa, Artificial Sequence
  Description of Artificial Sequence Synthetic peptide
GGGGSGGGGSGGGGSGGGGSGGGGS

SEQ ID NO: 3 - protein, 20 aa, Artificial Sequence
  Description of Artificial Sequence Synthetic peptide
GGGGGGGGGGGGGGGGGGGG

SEQ ID NO: 4 - protein, 25 aa, Artificial Sequence
  Description of Artificial Sequence Synthetic peptide
GGGGSGGGGSGGGGSGGGGSGGGGS

SEQ ID NO: 5 - protein, 246 aa, Artificial Sequence
  Description of Artificial Sequence Synthetic polypeptide
QVQLQESGPGLVKPSETLSLTCTVSGGSISPYYWTWIRQPPGKCLELIGYIHSSGYTDYNPSLKSRVTISGDTSKKQFSLKLSSVTAADTAVYYCARADWDRLRALDIWGQGTMVTVSSGGGGSGGGGSGGGGSGGGGSDIQLTQSPSSLSASVGDRVTITCRASQSIRSHLNWYQQKPGKAPKLLIYGASNLQSGVPSRFSGSGSGTDFTLTISSLQPEDFATYYCQQSTGAWNWFGCGTKVEIK

SEQ ID NO: 6 - protein, 710 aa, Artificial Sequence
  Description of Artificial Sequence Synthetic polypeptide
QVQLQESGPGLVKPSETLSLTCTVSGGSISPYYWTWIRQPPGKCLELIGYIHSSGYTDYNPSLKSRVTISGDTSKKQFSLKLSSVTAADTAVYYCARADWDRLRALDIWGQGTMVTVSSGGGGSGGGGSGGGGSGGGGSDIQLTQSPSSLSASVGDRVTITCRASQSIRSHLNWYQQKPGKAPKLLIYGASNLQSGVPSRFSGSGSGTDFTLTISSLQPEDFATYYCQQSTGAWNWFGCGTKVEIKGGGGSGGGGSEVQLLESGGGLVQPGGSLRLSCAASGFTFSSYAMNWVRQAPGKGLEWVSAITMSGITAYYTDDVKGRFTISRDNSKNTLYLQMNSLRAEDTAVYYCAKEEFLPGTHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, Artificial Sequence
  Description of Artificial Sequence Synthetic polypeptide
AIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 710 aa, Artificial Sequence
  Description of Artificial Sequence Synthetic polypeptide
EVQLLESGGGLVQPGGSLRLSCAASGFTFSSYAMNWVRQAPGKGLEWVSAITMSGITAYYTDDVKGRFTISRDNSKNTLYLQMNSLRAEDTAVYYCAKEEFLPGTHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQESGPGLVKPSETLSLTCTVSGGSISPYYWTWIRQPPGKCLELIGYIHSSGYTDYNPSLKSRVTISGDTSKKQFSLKLSSVTAADTAVYYCARADWDRLRALDIWGQGTMVTVSSGGGGSGGGGSGGGGSGGGGSDIQLTQSPSSLSASVGDRVTITCRASQSIRSHLNWYQQKPGKAPKLLIYGASNLQSGVPSRFSGSGSGTDFTLTISSLQPEDFATYYCQQSTGAWNWFGCGTKVEIK

SEQ ID NO: 9 - protein, 720 aa, Artificial Sequence [sequence omitted]

SEQ ID NO: 10 - DNA, 2130 bp, Artificial Sequence
  Description of Artificial Sequence Synthetic polynucleotide
caggtgcagctgcaggaatctggccctggcctcgtgaagccctccgagacactgtctctgacctgcaccgtgtccggcggctccatctccccttactactggacctggatcagacagccccctggcaagtgcctggaactgatcggctacatccactcctccggctacaccgactacaaccccagcctgaagtccagagtgaccatctccggcgacacctccaagaagcagttctccctgaagctgtcctccgtgaccgccgctgataccgccgtgtactactgcgccagagccgactgggacagactgagagccctggacatctggggccagggcacaatggtcaccgtgtctagcggaggcggaggatctggtggtggtggatctggcggcggaggaagtggtggcggaggctctgatatccagctgacccagtccccctccagcctgtctgcttctgtgggcgaccgcgtgaccatcacctgtagagcctcccagtccatccggtcccacctgaactggtatcagcagaagcccggcaaggcccccaagctgctgatctacggcgcctccaatctgcagtccggcgtgccctctagattctccggatctggctccggcaccgactttaccctgaccatcagctccctgcagcccgaggacttcgccacctactactgccagcagtctaccggcgcctggaattggttcggctgcggcaccaaggtggaaatcaagggcggagggggatccggcggagggggctctgaggtgcagctgttggagtctgggggaggcttggtacagcctggggggtccctgagactctcctgtgcagcctctggattcacctttagcagctatgccatgaactgggtccgccaggctccagggaaggggctggagtgggtctcagctattactatgagtggtattaccgcatactacaccgacgacgtgaagggccggttcaccatctccagagacaattccaagaacacgctatatctgcaaatgaacagcctgagggccgaggacacggccgtatattactgtgcgaaggaagaatttttacctggaacgcactactactacggtatggacgtctggggccaagggaccacggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 11 - DNA, 642 bp, Artificial Sequence
  Description of Artificial Sequence Synthetic polynucleotide
gccatccagatgacccagtctccatcctccctgtctgcatctgtaggagacagagtcaccatcacttgccgggcaagtcagggcattagaaatgatttaggctggtatcaacagaagccagggaaagcccctaaactcctgatctattctgcatccactttacaaagtggggtcccatcaaggttcagcggcagtggatctggcacagatttcactctcaccatcagcagcctgcagcctgaggattttgcaacttattactgtctacaagattacaattacccgtggacgttcggccaagggaccaaggttgaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 12 - DNA, 2130 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgccatgaactgggtccgccaggctccagggaaggggctggagtgggtctcagctattactatgagtggtattaccgcatactacaccgacgacgtgaagggccggttcaccatctccagagacaattccaagaacacgctatatctgcaaatgaacagcctgagggccgaggacacggccgtatattactgtgcgaaggaagaatttttacctggaacgcactactactacggtatggacgtctggggccaagggaccacggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaaggcggagggggatccggcggagggggctctcaggtgcagctgcaggaatctggccctggcctcgtgaagccctccgagacactgtctctgacctgcaccgtgtccggcggctccatctccccttactactggacctggatcagacagccccctggcaagtgcctggaactgatcggctacatccactcctccggctacaccgactacaaccccagcctgaagtccagagtgaccatctccggcgacacctccaagaagcagttctccctgaagctgtcctccgtgaccgccgctgataccgccgtgtactactgcgccagagccgactgggacagactgagagccctggacatctggggccagggcacaatggtcaccgtgtctagcggaggcggaggatctggtggtggtggatctggcggcggaggaagtggtggcggaggctctgatatccagctgacccagtccccctccagcctgtctgcttctgtgggcgaccgcgtgaccatcacctgtagagcctcccagtccatccggtcccacctgaactggtatcagcagaagcccggcaaggcccccaagctgctgatctacggcgcctccaatctgcagtccggcgtgccctctagattctccggatctggctccggcaccgactttaccctgaccatcagctccctgcagcccgaggacttcgccacctactactgccagcagtctaccggcgcctggaattggttcggctgcggcaccaaggtggaaatcaag

SEQ ID NO: 13 - DNA, 2160 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgccatgaactgggtccgccaggctccagggaaggggctggagtgggtctcagctattactatgagtggtattaccgcatactacaccgacgacgtgaagggccggttcaccatctccagagacaattccaagaacacgctatatctgcaaatgaacagcctgagggccgaggacacggccgtatattactgtgcgaaggaagaatttttacctggaacgcactactactacggtatggacgtctggggccaagggaccacggtcaccgtctcctcagcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtggcggagggggctctggcggagggggatcccaggtgcagctgcaggaatctggccctggcctcgtgaagccctccgagacactgtctctgacctgcaccgtgtccggcggctccatctccccttactactggacctggatcagacagccccctggcaagtgcctggaactgatcggctacatccactcctccggctacaccgactacaaccccagcctgaagtccagagtgaccatctccggcgacacctccaagaagcagttctccctgaagctgtcctccgtgaccgccgctgataccgccgtgtactactgcgccagagccgactgggacagactgagagccctggacatctggggccagggcacaatggtcaccgtgtctagcggaggcggaggatctggtggtggtggatctggcggcggaggaagtggtggcggaggctctgatatccagctgacccagtccccctccagcctgtctgcttctgtgggcgaccgcgtgaccatcacctgtagagcctcccagtccatccggtcccacctgaactggtatcagcagaagcccggcaaggcccccaagctgctgatctacggcgcctccaatctgcagtccggcgtgccctctagattctccggatctggctccggcaccgactttaccctgaccatcagctccctgcagcccgaggacttcgccacctactactgccagcagtctaccggcgcctggaattggttcggctgcggcaccaaggtggaaatcaagggcggaggtggctctggcggagggggatccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 14 - protein, 119 aa, Artificial Sequence
  Description of Artificial Sequence Synthetic polypeptide
EVQLLESGPGLVKPSETLSLTCNVAGGSISPYYWTWIRQPPGKGLELIGYIHSSGYTDYNPSLKSRVTISGDTSKKQFSLHVSSVTAADTAVYFCARGDWDLLHALDIWGQGTLVTVSS

SEQ ID NO: 15 - protein, 107 aa, Artificial Sequence
  Description of Artificial Sequence Synthetic polypeptide
EIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGGGTKLEIK

SEQ ID NO: 16 - protein, 119 aa, Artificial Sequence
  Description of Artificial Sequence Synthetic polypeptide
EVQLLESGPGLVKPSETLSLTCNVAGGSISPYYWTWIRQPPGKGLELIGYIHSSGYTDYNPSLKSRVTISGDTSKKQFSLHVSSVTAADTAVYFCARADWDLLHALDIWGQGTLVTVSS

SEQ ID NO: 17 - protein, 119 aa, Artificial Sequence
  Description of Artificial Sequence Synthetic polypeptide
EVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSS

SEQ ID NO: 18 - protein, 107 aa, Artificial Sequence
  Description of Artificial Sequence Synthetic polypeptide
EIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIK

SEQ ID NO: 19 - protein, 124 aa, Artificial Sequence
  Description of Artificial Sequence Synthetic polypeptide
EMQLLESGGGLVQPGGSLRLSCAASGFTFSSYAMNWVRQAPGEGLEWVSAITISGITAYYTDSVKGRFTISRDNSKNTLYLQMNSLRAGDTAVYYCAKEEFLPGTHYYYGMDVWGQGTTVTVSS

SEQ ID NO: 20 - protein, 107 aa, Artificial Sequence
  Description of Artificial Sequence Synthetic polypeptide
AIQMTQSPSSLSASVGDRVTITCRASQGIRNDLGWYQQKPGKAPKLVIYSASTLQSGVPSRFSGSGSGTDFTLSISSLQPDDFATYYCLQDYNYPWTFGQGTKVEIK

SEQ ID NO: 21 - protein, 107 aa, Artificial Sequence
  Description of Artificial Sequence Synthetic polypeptide
AIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIK

SEQ ID NO: 22 - DNA, 43 bp, Artificial Sequence
  Description of Artificial Sequence Synthetic primer
tggctccccggggcgcgctgtgaggtgcagctgttggagtcgg

SEQ ID NO: 23 - DNA, 30 bp, Artificial Sequence
  Description of Artificial Sequence Synthetic primer
ctccgccactcgagacggtgaccagggtcc

SEQ ID NO: 24 - DNA, 58 bp, Artificial Sequence
  Description of Artificial Sequence Synthetic primer
accgtctcgagtggcggagggggctctgggggagggggcagcggcggcggaggatctg

SEQ ID NO: 25 - DNA, 59 bp, Artificial Sequence
  Description of Artificial Sequence Synthetic primer
agcggcggcggaggatctgggggagggggcagcgaaattgtgttgacacagtctccatc

SEQ ID NO: 26 - DNA, 51 bp, Artificial Sequence
  Description of Artificial Sequence Synthetic primer
gccccctccgccggatccccctccgcctttgatctccagcttggtccctcc

SEQ ID NO: 27 - DNA, 45 bp, Artificial Sequence
  Description of Artificial Sequence Synthetic primer
ttctctccacaggtgtacactccgaggtgcagctgttggagtcgg

SEQ ID NO: 28 - DNA, 30 bp, Artificial Sequence; identical to SEQ ID NO: 23
  Description of Artificial Sequence Synthetic primer

SEQ ID NO: 29 - DNA, 57 bp, Artificial Sequence
  Description of Artificial Sequence Synthetic primer
gtaaaggcggagggggatccggcggagggggctctgaggtgcagctgttggagtcgg

SEQ ID NO: 30 - DNA, 49 bp, Artificial Sequence
  Description of Artificial Sequence Synthetic primer
gatcaatgaattcgcggccgctcatttgatctccagcttggtccctccg

SEQ ID NO: 31 - DNA, 44 bp, Artificial Sequence
  Description of Artificial Sequence Synthetic primer
tggctccccggggcgcgctgtgccatccagatgacccagtctcc

SEQ ID NO: 32 - DNA, 42 bp, Artificial Sequence
  Description of Artificial Sequence Synthetic primer
tggtgcagccaccgtacgtttgatttcaaccttggtcccttg

SEQ ID NO: 33 - DNA, 55 bp, Artificial Sequence
  Description of Artificial Sequence Synthetic primer
aaggcggagggggatccggcggagggggctctgagatgcagctgttggagtctgg

SEQ ID NO: 34 - DNA, 43 bp, Artificial Sequence
  Description of Artificial Sequence Synthetic primer
gatgggcccttggtcgacgctgaggagacggtgaccgtggtcc

SEQ ID NO: 35 - DNA, 21 bp, Artificial Sequence
  Description of Artificial Sequence Synthetic primer
caactccaggcacttccctgg

SEQ ID NO: 36 - DNA, 21 bp, Artificial Sequence
  Description of Artificial Sequence Synthetic primer
ccagggaagtgcctggagttg

SEQ ID NO: 37 - DNA, 22 bp, Artificial Sequence
  Description of Artificial Sequence Synthetic primer
gtcccaatcggctctcgcacag

SEQ ID NO: 38 - DNA, 22 bp, Artificial Sequence
  Description of Artificial Sequence Synthetic primer
ctgtgcgagagccgattgggac

SEQ ID NO: 39 - DNA, 53 bp, Artificial Sequence
  Description of Artificial Sequence Synthetic primer
caatgaattcgcggccgctcatttgatctccagcttggtcccacagccgaaag

SEQ ID NO: 40 - protein, 5 aa, Artificial Sequence
  Description of Artificial Sequence Synthetic peptide
GGGGS

SEQ ID NO: 41 - protein, 256 aa, Artificial Sequence
  Description of Artificial Sequence Synthetic polypeptide
GGGGSGGGGSEVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSSGGGGSGGGGSGGGGSGGGGSEIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIK

SEQ ID NO: 42 - protein, 380 aa, Artificial Sequence
  Description of Artificial Sequence Synthetic polypeptide
EVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSSGGGGSGGGGSGGGGSGGGGSEIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIKGGGGSGGGGSEMQLLESGGGLVQPGGSLRLSCAASGFTFSSYAMNWVRQAPGEGLEWVSAITISGITAYYTDSVKGRFTISRDNSKNTLYLQMNSLRAGDTAVYYCAKEEFLPGTHYYYGMDVWGQGTTVTVSS

SEQ ID NO: 43 - DNA, 58 bp, Artificial Sequence
  Description of Artificial Sequence Synthetic primer
ccccctccgccggatccccctccgcctttgatctccagcttggtcccacagccgaaag

SEQ ID NO: 44 - protein, 21 aa, Artificial Sequence
  Description of Artificial Sequence Synthetic peptide
KVDKRVEPKSCGGGGSGGGGS

SEQ ID NO: 45 - protein, 25 aa, Artificial Sequence
  Description of Artificial Sequence Synthetic peptide
GGGGSGGGGSDKTHTCPPCPAPELL

SEQ ID NO: 46 - protein, 292 aa, Artificial Sequence
  Description of Artificial Sequence Synthetic polypeptide
KVDKRVEPKSCGGGGSGGGGSEVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSSGGGGSGGGGSGGGGSGGGGSEIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIKGGGGSGGGGSDKTHTCPPCPAPELL

SEQ ID NO: 47 - DNA, 28 bp, Artificial Sequence
  Description of Artificial Sequence Synthetic primer
cacggtcaccgtctcctcagcgtcgacc

SEQ ID NO: 48 - DNA, 49 bp, Artificial Sequence
  Description of Artificial Sequence Synthetic primer
ccctccgccagagccccctccgccacaagatttgggctcaactctcttg

SEQ ID NO: 49 - DNA, 49 bp, Artificial Sequence
  Description of Artificial Sequence Synthetic primer
gagggggctctggcggagggggatccgacaaaactcacacatgcccacc

SEQ ID NO: 50 - DNA, 28 bp, Artificial Sequence
  Description of Artificial Sequence Synthetic primer
tcaatgaattcgcggccgctcatttacc

SEQ ID NO: 51 - DNA, 24 bp, Artificial Sequence
  Description of Artificial Sequence Synthetic primer
gaggtgcagctgttggagtcgggc

SEQ ID NO: 52 - DNA, 65 bp, Artificial Sequence
  Description of Artificial Sequence Synthetic primer
gtgtgagttttgtcggatccccctccgccagagccacctccgcctttgatctccagcttggtccc

SEQ ID NO: 53 - DNA, 42 bp, Artificial Sequence
  Description of Artificial Sequence Synthetic primer
tctggcggagggggatcccaggtgcagctgcaggaatctggc

SEQ ID NO: 54 - DNA, 63 bp, Artificial Sequence
  Description of Artificial Sequence Synthetic primer
gtgagttttgtcggatccccctccgccagagccacctccgcccttgatttccaccttggtgcc

SEQ ID NO: 55 - DNA, 45 bp, Artificial Sequence
  Description of Artificial Sequence Synthetic primer
ttctctccacaggtgtacactcccaggtgcagctgcaggaatctg

SEQ ID NO: 56 - DNA, 45 bp, Artificial Sequence
  Description of Artificial Sequence Synthetic primer
cctccgccggatccccctccgcccttgatttccaccttggtgccg

SEQ ID NO: 57 - DNA, 47 bp, Artificial Sequence
  Description of Artificial Sequence Synthetic primer
gggggatccggcggagggggctctgaggtgcagctgttggagtctgg

SEQ ID NO: 58 - DNA, 55 bp, Artificial Sequence
  Description of Artificial Sequence Synthetic primer
aaaggcggagggggatccggcggagggggctctcaggtgcagctgcaggaatctg

SEQ ID NO: 59 - DNA, 45 bp, Artificial Sequence
  Description of Artificial Sequence Synthetic primer
tcaatgaattcgcggccgctcacttgatttccaccttggtgccgc

SEQ ID NO: 60 - DNA, 57 bp, Artificial Sequence; identical to SEQ ID NO: 29
  Description of Artificial Sequence Synthetic primer

SEQ ID NO: 61 - protein, 20 aa, Artificial Sequence
  Description of Artificial Sequence Synthetic peptide
GGGGSGGGGSGGGGSGGGGS

SEQ ID NO: 62 - DNA, 66 bp, Artificial Sequence
  Description of Artificial Sequence Synthetic primer
gatcctccgccgccgctgccccctcccccagagccccctccgccactcgagacggtgaccagggtc

SEQ ID NO: 63 - DNA, 62 bp, Artificial Sequence
  Description of Artificial Sequence Synthetic primer
agggggcagcggcggcggaggatctgggggagggggcagcgaaattgtgttgacacagtctc

SEQ ID NO: 64 - DNA, 44 bp, Artificial Sequence
  Description of Artificial Sequence Synthetic primer
caatgaattcgcggccgctcatttgatctccagcttggtcccac

SEQ ID NO: 65 - DNA, 45 bp, Artificial Sequence; identical to SEQ ID NO: 27
  Description of Artificial Sequence Synthetic primer

SEQ ID NO: 66 - DNA, 53 bp, Artificial Sequence
  Description of Artificial Sequence Synthetic primer
ggcggagggggatccggcggagggggctctgagatgcagctgttggagtctgg

SEQ ID NO: 67 - DNA, 41 bp, Artificial Sequence
  Description of Artificial Sequence Synthetic primer
atgggcccttggtcgacgctgaggagacggtgaccgtggtc

SEQ ID NO: 68 - protein, 246 aa, Artificial Sequence
  Description of Artificial Sequence Synthetic polypeptide
EVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSSGGGGSGGGGSGGGGSGGGGSEIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIK

SEQ ID NO: 69 - DNA, 24 bp, Artificial Sequence
  Description of Artificial Sequence Synthetic primer
gaggtgcagctgttggagtcgggc

SEQ ID NO: 70 - protein, 720 aa, Artificial Sequence
  Description of Artificial Sequence Synthetic polypeptide
EMQLLESGGGLVQPGGSLRLSCAASGFTFSSYAMNWVRQAPGEGLEWVSAITISGITAYYTDSVKGRFTISRDNSKNTLYLQMNSLRAGDTAVYYCAKEEFLPGTHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSEVQLLESGPGLVKPSETLSLTCNVAGGSISPYYWTWIRQPPGKCLELIGYIHSSGYTDYNPSLKSRVTISGDTSKKQFSLHVSSVTAADTAVYFCARADWDLLHALDIWGQGTLVTVSSGGGGSGGGGSGGGGSGGGGSEIVLTQSPSSLSTSVGDRVTITCRASQSIRSHLNWYQQKPGKAPKLLIYGASNLQSGVPSRFSGSGSGTDFTLTISSLQPEDFATYYCQQSYSFPLTFGCGTKLEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2017/0183397 A1
(43) Pub. Date: Jun. 29, 2017

(54) MULTI-SPECIFIC ANTI-PSEUDOMONAS PSL AND PCRV BINDING MOLECULES AND USES THEREOF

(71) Applicants: MEDIMMUNE, LLC, GAITHERSBURG, MD (US); MEDIMMUNE LIMITED, CAMBRIDGE (GB)

(72) Inventors: Antonio DIGIANDOMENICO, Gaithersburg, MD (US); Paul WARRENER, Gaithersburg, MD (US); Charles STOVER, Gaithersburg, MD (US); Bret SELLMAN, Gaithersburg, MD (US); Ralph MINTER, Cambridge (UK); Sandrine GUILLARD, Cambridge (UK); Steven RUST, Cambridge (UK); Mladen TOMICH, Exton, PA (US); Vignesh VENKATRAMAN, Cambridge (UK); Reena VARKEY, Gaithersburg, MD (US); Li PENG, Gaithersburg, MD (US); Melissa DAMSCHRODER, Gaithersburg, MD (US); Partha CHOWDHURY, Gaithersburg, MD (US); Nazzareno DIMASI, Gaithersburg, MD (US); Ryan FLEMING, Gaithersburg, MD (US); Binyam BEZABEH, Gaithersburg, MD (US); Changshou GAO, Gaithersburg, MD (US); Godfrey RAINEY, Gaithersburg, MD (US); Cuihua GAO, Gaithersburg, MD (US)

(21) Appl. No.: 15/308,635

(22) PCT Filed: May 4, 2015
(86) PCT No.: PCT/US15/29063

§ 371 Date: Nov. 3, 2016

Related U.S. Application Data
(60) Provisional application No. 61/988,669, filed on May 5, 2014.

(51) Int. Cl. C07K 16/12; A61K 45/06; A61K 39/40

(52) U.S. Cl. CPC C07K 16/1214; A61K 39/40; A61K 45/06; C07K 2317/31; C07K 2317/622; C07K 2319/00; A61K 2039/505

(57) ABSTRACT

This disclosure relates to combination therapies comprising anti-Pseudomonas Psl and PcrV bispecific binding molecules and related compositions, for use in prevention and treatment of Pseudomonas infection.

### BACKGROUND

### Sequence Listing

The instant application contains a Sequence Listing which has been submitted electronically in ASCII format and is hereby incorporated by reference in its entirety. Said ASCII copy, created on Apr. 14, 2015, is named PSEUD-104WO1_SL.txt and is 71,044 bytes in size.

### FIELD OF THE DISCLOSURE

This disclosure relates to combination therapies using anti-Pseudomonas Psl and PcrV binding domains, e.g., bispecific anti-Pseudomonas Psl and PcrV binding molecules, for use in the prevention and treatment of Pseudomonas infection. Furthermore, the disclosure provides compositions useful in such therapies.

### BACKGROUND OF THE DISCLOSURE

Pseudomonas aeruginosa (P. aeruginosa) is a gram-negative opportunistic pathogen that causes both acute and chronic infections in compromised individuals (Ma et al., Journal of Bacteriology 189(22):8353-8356 (2007)). This is partly due to the high innate resistance of the bacterium to clinically used antibiotics, and partly due to the formation of highly antibiotic-resistant biofilms (Drenkard E., Microbes Infect 5:1213-1219 (2003); Hancokc & Speert, Drug Resist Update 3:247-255 (2000)).

P. aeruginosa is a common cause of hospital-acquired infections in the Western world. It is a frequent causative agent of bacteremia in burn victims and immune compromised individuals (Lyczak et al., Microbes Infect 2:1051-1060 (2000)). It is also the most common cause of nosocomial gram-negative pneumonia (Craven et al., Semin Respir Infect 11:32-53 (1996)), especially in mechanically ventilated patients, and is the most prevalent pathogen in the lungs of individuals with cystic fibrosis (Pier et al., ASM News 6:339-347 (1998)).

Pseudomonas Psl exopolysaccharide is reported to be anchored to the surface of P. aeruginosa and is thought to be important in facilitating colonization of host tissues and in establishing/maintaining biofilm formation (Jackson, K. D., et al., J Bacteriol 186, 4466-4475 (2004)). Its structure comprises mannose-rich repeating pentasaccharide (Byrd, M. S., et al., Mol Microbiol 73, 622-638 (2009)).

PcrV is a relatively conserved component of the type III secretion system. PcrV appears to be an integral component of the translocation apparatus of the type III secretion system mediating the delivery of the type III secretory toxins into target eukaryotic cells (Sawa T., et al. Nat. Med. 5, 392-398 (1999)). Active and passive immunization against PcrV improved acute lung injury and mortality of mice infected with cytotoxic P. aeruginosa (Sawa et al. 2009). The major effect of immunization against PcrV was due to the blockade of translocation of the type III secretory toxins into eukaryotic cells.

Due to increasing multidrug resistance, there remains a need in the art for the development of novel strategies for the identification of new Pseudomonas-specific prophylactic and therapeutic agents.

### BRIEF SUMMARY

The disclosure provides a bispecific antibody comprising a binding domain that binds to Pseudomonas Psl and a binding domain that binds to Pseudomonas PcrV. In certain aspects, the Psl binding domain comprises a scFv fragment and the PcrV binding domain comprises an intact immunoglobulin. In certain aspects, the Psl binding domain comprises an intact immunoglobulin and the PcrV binding domain comprises a scFv fragment. In certain aspects, the bispecific antibody comprises a Bs-2 molecule, wherein the scFv is fused to the amino-terminus of the VH region of the intact immunoglobulin. In certain aspects, the bispecific antibody comprises a Bs-3 molecule, wherein the scFv is fused to the carboxy-terminus of the CH3 region of the intact immunoglobulin. In certain aspects, the bispecific antibody comprises a Bs-4 molecule, wherein the scFv is inserted in the hinge region of the intact immunoglobulin.

In certain aspects, the anti-Psl binding domain specifically binds to the same Pseudomonas Psl epitope as an antibody or antigen-binding fragment thereof comprising the heavy chain variable region (VH) and light chain variable region (VL) region at least 90% identical to the corresponding region of WapR-004. In certain aspects the anti-Psl binding domain specifically binds to Pseudomonas Psl, and competitively inhibits Pseudomonas Psl binding by an antibody or antigen-binding fragment thereof comprising a VH and VL region at least 90% identical to the corresponding region of WapR-004. In certain aspects, the WapR-004 VH and VL are arranged as a ScFv.

In certain aspects, the anti-PcrV binding domain specifically binds to the same Pseudomonas PcrV epitope as an antibody or antigen-binding fragment thereof comprising the VH and VL region of V2L2. In certain aspects, the anti-PcrV binding domain specifically binds to Pseudomonas PcrV, and competitively inhibits Pseudomonas PcrV binding by an antibody or antigen-binding fragment thereof comprising the VH and VL of V2L2. In certain aspects the anti-PcrV binding domain which specifically binds to the same Pseudomonas PcrV epitope comprises VH and VL regions at least 90% identical to the corresponding regions of V2L2.

In certain aspects the bispecific antibody comprises Bs2-GLO. In certain aspects the bispecific antibody comprises Bs3-GLO. In certain aspects the bispecific antibody comprises Bs4-GLO.

The disclosure further provides a cell comprising or producing the bispecific antibody as described above.

The disclosure further provides an isolated polynucleotide molecule comprising a polynucleotide that encodes the bispecific antibody as described above. The disclosure also provides a vector comprising the polynucleotide described above, and a cell comprising the polynucleotide or the vector.

The disclosure further provides a composition comprising the bispecific antibody provided herein and a pharmaceutically acceptable carrier.

In certain aspects, the bispecific antibody provided herein can be conjugated to an agent selected from the group consisting of antimicrobial agent, a therapeutic agent, a prodrug, a peptide, a protein, an enzyme, a lipid, a biological response modifier, pharmaceutical agent, a lymphokine, a heterologous antibody or fragment thereof, a detectable label, polyethylene glycol (PEG), and a combination of two or more of any said agents. In certain aspects the detectable label can be an enzyme, a fluorescent label, a chemiluminescent label, a bioluminescent label, a radioactive label, or a combination of two or more of the detectable labels.

The composition as provided herein can further comprise an antibiotic, e.g., Ciprofloxacin, Meropenem, or a combination thereof.

The disclosure further provides a method of preventing or treating a Pseudomonas infection in a subject in need thereof, comprising administering to a subject, e.g., a human subject, an effective amount of a bispecific antibody as provided herein, wherein the administration provides a therapeutic effect in the prevention or treatment of the Pseudomonas infection in the subject. In certain aspects the bispecific antibody can be administered for two or more prevention/treatment cycles. In certain aspects the Pseudomonas infection is a P. aeruginosa infection. In certain aspects, the infection can be an ocular infection, a lung infection, a burn infection, a wound infection, a skin infection, a blood infection, a bone infection, or a combination of two or more of said infections. In certain aspects the subject has acute pneumonia, burn injury, corneal infection, cystic fibrosis, or a combination thereof.

### BRIEF DESCRIPTION OF THE DRAWINGS/FIGURES

FIG. 1 (A-B): Schematic representation of (A) Bs1-TNFα/W4, Bs2-TNFα/W4, Bs3-TNFα/W4 and (B) Bs2-V2L2/W4-RAD, Bs3-V2L2/W4-RAD, and Bs4-V2L2-W4-RAD Psl/PcrV bispecific antibodies. (A) For Bs1-TNFα/W4, the W4 scFv is fused to the amino-terminus of TNFα VL through a (G4S)2 linker (SEQ ID NO: 1). For Bs2-TNFα/W4, the W4 scFv is fused to the amino-terminus of TNFα VH through a (G4S)2 linker (SEQ ID NO: 1). For Bs3-TNFα/W4, the W4 scFv is fused to the carboxy-terminus of CH3 through a (G4S)2 linker (SEQ ID NO: 1). (B) For Bs2-V2L2-2C, the W4-RAD scFv is fused to the amino-terminus of V2L2 VH through a (G4S)2 linker (SEQ ID NO: 1). For Bs2-W4-RAD-2C, the V2L2 scFv is fused to the amino-terminus of W4-RAD VH through a (G4S)2 linker (SEQ ID NO: 1). For Bs3-V2L2-2C, the W4-RAD scFv is fused to the carboxy-terminus of CH3 through a (G4S)2 linker (SEQ ID NO: 1). For Bs4-V2L2-2C, the W4-RAD scFv is inserted in the hinge region, linked by (G4S)2 linker (SEQ ID NO: 1) on the N-terminal and C-terminal of the scFv. FIG. 1B discloses “(G₄S)₄” as SEQ ID NO: 61.

FIG. 2A shows that bispecific antibodies Bs2-GLO (closed triangles), Bs3-GLO (closed squares) and Bs4-GLO (closed diamonds) block the attachment of P. aeruginosa to cultured epithelial cells. FIG. 2B shows the statistical analysis. The AUC for each antibody activity response curve was calculated using the linear trapezoidal rule on the means at different concentrations in the log scale. AUC calculation and statistical comparisons between different antibodies were performed using PK package in R software.

FIG. 3A shows that bispecific antibodies Bs2-GLO (closed triangles), Bs3-GLO (closed squares) and Bs4-GLO (closed diamonds) inhibit P. aeruginosa-induced lysis of lung epithelial cells. FIG. 3B shows the statistical analysis. The AUC for each antibody activity response curve was calculated using the linear trapezoidal rule on the means at different concentrations in the log scale. AUC calculation and statistical comparisons between different antibodies were performed using PK package in R software.

### DETAILED DESCRIPTION

### I. Definitions

It is to be noted that the term “a” or “an” entity refers to one or more of that entity; for example, “a binding molecule, e.g., a bispecific antibody, that specifically binds to Pseudomonas Psl and PcrV,” is understood to represent one or more binding molecules that specifically bind to Pseudomonas Psl and PcrV. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. An “isolated” polypeptide can be materially changed from a naturally-occurring polypeptide such that the polypeptide is “non-naturally-occurring.” Recombinantly produced polypeptides and proteins expressed, e.g., from polynucleotides inserted into heterologous vectors, or contained in heterologous host cells, are considered “isolated,” and “non-naturally-occurring” as disclosed herein.

Other polypeptides disclosed herein are fragments, derivatives, analogs, or variants of the foregoing polypeptides, and any combination thereof. The terms “fragment,” “variant,” “derivative” and “analog” when referring to a binding molecule such as a bispecific antibody that specifically binds to Pseudomonas Psl and PcrV as disclosed herein include any polypeptides that retain at least some of the antigen-binding properties of the corresponding native antibody or polypeptide. Fragments of polypeptides include, for example, proteolytic fragments, as well as deletion fragments, in addition to specific antibody fragments discussed elsewhere herein. Variants of a binding molecule, e.g., a bispecific antibody that specifically binds to Pseudomonas Psl and PcrV as disclosed herein include fragments as described above, and also polypeptides with altered amino acid sequences due to amino acid substitutions, deletions, or insertions. Variants can occur naturally or be non-naturally occurring. Non-naturally occurring variants can be produced using art-known mutagenesis techniques. Variant polypeptides can comprise conservative or non-conservative amino acid substitutions, deletions or additions. Derivatives of a binding molecule, e.g., a bispecific antibody that specifically binds to Pseudomonas Psl and PcrV as disclosed herein are polypeptides that have been altered so as to exhibit additional features not found on the native polypeptide. Examples include fusion proteins. Variant polypeptides can also be referred to herein as “polypeptide analogs.” As used herein a “derivative” of a binding molecule, e.g., a bispecific antibody that specifically binds to Pseudomonas Psl and PcrV refers to a subject polypeptide having one or more residues chemically derivatized by reaction of a functional side group. Also included as “derivatives” are those peptides that contain one or more naturally occurring amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, that has been removed from its native environment. For example, a recombinant polynucleotide encoding a binding molecule, e.g., a bispecific antibody that specifically binds to Pseudomonas Psl and PcrV contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid that consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding an a binding molecule, e.g., a bispecific antibody, that specifically binds to Pseudomonas Psl and PcrV, e.g., an antibody, or antigen-binding fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide as disclosed herein, e.g., a polynucleotide encoding a binding molecule, e.g., a bispecific antibody, that specifically binds to Pseudomonas Psl and PcrV, e.g., an antibody, or antigen-binding fragment, variant, or derivative thereof. According to the signal hypothesis, proteins secreted by mammalian cells have a signal peptide or secretory leader sequence that is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

Disclosed herein are certain binding molecules, or antigen-binding fragments, variants, or derivatives thereof. Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that can specifically bind a given antigenic determinant, or epitope. A non-limiting example of an binding molecule is a bispecific antibody or fragment thereof that comprises at least two distinct binding domains that specifically bind different antigenic determinants or epitopes. In certain aspects, a bispecific antibody as provided herein can be said to comprise a first binding domain binding to a first epitope, and a second binding domain binding to a second epitope.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. This quaternary binding molecule structure forms the antigen binding site present at the end of each arm of the Y. More specifically, the antigen binding site is defined by three CDRs on each of the VH and VL chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in a binding molecule, e.g., a bispecific antibody, that specifically binds to Pseudomonas Psl and PcrV, e.g, an antibody, or antigen-binding fragment, variant, or derivative thereof as disclosed herein are according to the Kabat numbering system.

Binding molecules, e.g., bispecific antibodies or antigen-binding fragments, variants, or derivatives thereof include, but are not limited to, polyclonal, monoclonal, human, humanized, or chimeric antibodies, single chain antibodies, multispecific antibodies, e.g., bispecific antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019 Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that a binding molecule, e.g., an antibody or fragment, variant, or derivative thereof binds to an epitope via an antigen binding domain, and that the binding entails some complementarity between an antigen binding domain and the epitope. A binding molecule as provided herein can contain one, two, three, four, or more binding domains that can be the same or different, and that can bind to the same epitope, or to two or more different epitopes. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” may be deemed to have a higher specificity for a given epitope than binding molecule “B,” or binding molecule “A” may be said to bind to epitope “C” with a higher specificity than it has for related epitope “D.”

By “preferentially binds,” it is meant that the antibody specifically binds to an epitope via a binding domain more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, a binding molecule, e.g., an antibody can be considered to bind a first epitope preferentially if it binds said first epitope with a dissociation constant (K_{D}) that is less than the antibody's K_{D} for the second epitope. In another non-limiting example, a binding molecule such as an antibody can be considered to bind a first antigen preferentially if it binds the first epitope with an affinity that is at least one order of magnitude less than the antibody's K_{D} for the second epitope. In another non-limiting example, a binding molecule can be considered to bind a first epitope preferentially if it binds the first epitope with an affinity that is at least two orders of magnitude less than the antibody's K_{D} for the second epitope.

In another non-limiting example, a binding molecule, e.g., an antibody or fragment, variant, or derivative thereof can be considered to bind a first epitope preferentially if it binds the first epitope with an off rate (k(off)) that is less than the antibody's k(off) for the second epitope. In another non-limiting example, a binding molecule can be considered to bind a first epitope preferentially if it binds the first epitope with an affinity that is at least one order of magnitude less than the antibody's k(off) for the second epitope. In another non-limiting example, a binding molecule can be considered to bind a first epitope preferentially if it binds the first epitope with an affinity that is at least two orders of magnitude less than the antibody's k(off) for the second epitope.

A binding molecule, e.g., an antibody or fragment, variant, or derivative thereof disclosed herein can be said to bind a target antigen, e.g., a polysaccharide or polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. A binding molecule as disclosed herein can be said to bind a target antigen, e.g., a polysaccharide or a polypeptide, with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen, e.g., a polysaccharide or a polypeptide, with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. A binding molecule as disclosed herein can be said to bind a target antigen, e.g., a polysaccharide or a polypeptide, with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with a binding domain of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹° M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibody fragments including single-chain antibodies can comprise the variable region(s) alone or in combination with the entirety or a portion of the following: hinge region, CH1, CH2, and CH3 domains. Also included are antigen-binding fragments also comprising any combination of variable region(s) with a hinge region, CH1, CH2, and CH3 domains. Binding molecules, e.g., bispecific antibodies, or antigen-binding fragments thereof disclosed herein can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. a binding molecule, e.g., an antibody comprising a heavy chain portion comprises at least one of: a CH1 domain, a hinge (e.g., upper, middle, and/or lower hinge region) domain, a CH2 domain, a CH3 domain, or a variant or fragment thereof. For example, a binding molecule, e.g., an antibody or fragment, variant, or derivative thereof can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a binding molecule, e.g., an antibody or fragment, variant, or derivative thereof comprises a polypeptide chain comprising a CH3 domain. Further, a binding molecule for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

The heavy chain portions of a binding molecule, e.g., an antibody as disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion comprises at least one of a VL or CL domain.

Binding molecules, e.g., bispecific antibodies or antigen-binding fragments, variants, or derivatives thereof disclosed herein can be described or specified in terms of the epitope(s) or portion(s) of an antigen, e.g., a target polysaccharide or a polypeptide that they recognize or specifically bind. The portion of a target antigen that specifically interacts with the antigen binding domain of an antibody is an “epitope,” or an “antigenic determinant.” A target antigen, e.g., a polysaccharide or a polypeptide, can comprise a single epitope, but typically comprises at least two epitopes, and can include any number of epitopes, depending on the size, conformation, and type of antigen.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (that can be intact, partial or modified) is obtained from a second species. In some embodiments the target binding region or site will be from a non-human source (e.g. mouse or primate) and the constant region is human.

The terms “multispecific antibody” or “bispecific antibody” as used herein refer to an antibody that has binding domains specific for two or more different antigens or epitopes within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)).

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., anti-Pseudomonas Psl and PcrV bispecific antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change, infection, or disorder. Beneficial or desired clinical results include, but are not limited to, alleviation of symptoms, diminishment of extent of disease, stabilized (i.e., not worsening) state of disease, clearance or reduction of an infectious agent such as P. aeruginosa in a subject, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the infection, condition, or disorder as well as those prone to have the condition or disorder or those in which the condition or disorder is to be prevented, e.g., in burn patients or immunosuppressed patients susceptible to P. aeruginosa infection.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on.

As used herein, phrases such as “a subject that would benefit from administration of an anti-Pseudomonas Psl and PcrV bispecific binding molecule” and “an animal in need of treatment” includes subjects, such as mammalian subjects, that would benefit from administration of an anti-Pseudomonas Psl and PcrV bispecific binding molecule, such as a bispecific antibody. Such binding molecules can be used, e.g., for detection of Pseudomonas Psl or PcrV (e.g., for a diagnostic procedure) and/or for treatment, i.e., palliation or prevention of a disease, with anti-Pseudomonas Psl and PcrV bispecific binding molecules. As described in more detail herein, the anti-Pseudomonas Psl and PcrV bispecific binding molecules can be used in unconjugated form or can be conjugated, e.g., to a drug, prodrug, or an isotope.

The term “synergistic effect”, as used herein, refers to a greater-than-additive therapeutic effect produced by a combination of compounds wherein the therapeutic effect obtained with the combination exceeds the additive effects that would otherwise result from individual administration the compounds alone. Certain embodiments include methods of producing a synergistic effect in the treatment of Pseudomonas infections, wherein said effect is at least 5%, at least 10%, at least 20%, at least 30%, at least 40%, at least 50%, at least 60%, at least 70%, at least 80%, at least 90%, at least 100%, at least 200%, at least 500%, or at least 1000% greater than the corresponding additive effect.

### II. Binding Domains and Binding Molecules

Antibodies that bind Psl and formats for using these antibodies have been described in the art. See, for example, International Application Nos. PCT/US2012/041538, filed Jun. 8, 2012, published as WO2012/170807 on Dec. 13, 2012 and PCT/US2012/63639, filed Nov. 6, 2012, published as WO2013/070565 on May 16, 2013 (attorney docket no. AEMS-115WO1, entitled “MULTISPECIFIC AND MULTIVALENT BINDING PROTEINS AND USES THEREOF”), which are herein incorporated in their entireties by reference.

This disclosure provides bispecific binding molecules, wherein one binding domain specifically binds Psl and the other binding domain specifically binds PcrV, and wherein administration of the binding molecules results in beneficial or synergistic effects against Pseudomonas infections by (a) inhibiting attachment of Pseudomonas aeruginosa to epithelial cells, (b) promoting, mediating, or enhancing opsonophagocytic killing (OPK) of P. aeruginosa, (c) inhibiting attachment of P. aeruginosa to epithelial cells, or (d) disrupting the activity of the type III toxin secretion system.

In one embodiment, the bispecific binding molecule comprises a first binding domain that specifically binds to the same Pseudomonas Psl epitope as an antibody or antigen-binding fragment thereof comprising the heavy chain variable region (VH) and light chain variable region (VL) region of WapR-004, W4-RAD, or W4-RAD-2C, and a second binding domain specifically binds to the same Pseudomonas PcrV epitope as an antibody or antigen binding fragment thereof comprising the heavy chain variable region (VH) and light chain variable region (VL) of V2L2.

In one embodiment, the composition comprises a first binding domain that specifically binds to Pseudomonas Psl and/or competitively inhibits Pseudomonas Psl binding by an antibody or antigen-binding fragment thereof comprising the VH and VL of WapR-004, W4-RAD, or W4-RAD-2C, and a second binding domain specifically binds to the same Pseudomonas PcrV epitope and/or competitively inhibits Pseudomonas PcrV binding by an antibody or antigen binding fragment thereof comprising the heavy chain variable region (VH) and light chain variable region (VL) of V2L2.

Methods of making antibodies are well known in the art and described herein. Once antibodies to various fragments of, or to the full-length Pseudomonas Psl or PcrV without the signal sequence, have been produced, determining which amino acids, or epitope, of Pseudomonas Psi or PcrV to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols can be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

In certain aspects, the disclosure is directed to a bispecific binding molecule, e.g., a bispecific antibody or fragment, variant, or derivative thereof that specifically binds to Pseudomonas Psi and PcrV with affinities characterized by dissociation constants (K_{D}) that are less than the K_{Ds} of certain reference monoclonal antibodies.

In certain embodiments an anti-Pseudomonas Psi and PcrV bispecific binding molecule, e.g., a bispecific antibody or antigen-binding fragment, variant or derivative thereof as disclosed herein can bind specifically to at least one epitope of both Psi and PcrV, i.e., binds to such epitopes more readily than it would bind to unrelated, or random epitopes; binds preferentially to at least one epitope of both Psi and PcrV, i.e., binds to such epitopes more readily than it would bind to related, similar, homologous, or analogous epitopes; competitively inhibits binding of a reference antibody that itself binds specifically or preferentially to certain epitopes of both Psi and PcrV; or binds to at least one epitope each of Psi and PcrV with an affinity characterized, independently, by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M about 10⁻¹⁰ M about 5×10⁻¹¹ M, about 10⁻¹¹ M, about 5×10⁻¹²M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵M.

As used in the context of binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments a bispecific binding molecule, e.g., a bispecific antibody, or antigen-binding fragment, variant, or derivative thereof binds to both Pseudomonas Psl and PcrV with an off rate (k(off)) that is independently less than or equal to 5×10⁻² sec⁻¹, 10⁻² sec⁻¹, 5×10⁻³ sec⁻¹ or 10⁻³ sec⁻¹. Alternatively, an antibody, or antigen-binding fragment, variant, or derivative thereof binds Pseudomonas Psl and PcrV with an off rate (k(off)) that is independently less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, a bispecific binding molecule, e.g., a bispecific antibody, or antigen-binding fragment, variant, or derivative thereof as disclosed herein can bind both Pseudomonas Psl and PcrV with an on rate (k(on)) independently greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, a bispecific binding molecule, e.g., a bispecific antibody, or antigen-binding fragment, variant, or derivative thereof as disclosed herein can bind Pseudomonas Psl and PcrV with an on rate (k(on)) independently greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

In various embodiments, an anti-Pseudomonas Psl and PcrV bispecific binding molecule, e.g., a bispecific antibody, or antigen-binding fragment, variant, or derivative thereof as described herein can promote opsonophagocytic killing of Pseudomonas, or can inhibit Pseudomonas binding to epithelial cells. In certain embodiments described herein, the Pseudomonas Psl and PcrV targets are Pseudomonas aeruginosa Psl or PcrV. In other embodiments, certain binding molecules described herein can bind to structurally related polysaccharide molecules regardless of their source. Such Psl-like molecules would be expected to be identical to or have sufficient structural relatedness to P. aeruginosa Psl to permit specific recognition by one or more of the binding molecules disclosed. In other embodiments, certain binding molecules described herein can bind to structurally related polypeptide molecules regardless of their source. Such PcrV-like molecules would be expected to be identical to or have sufficient structural relatedness to P. aeruginosa PcrV to permit specific recognition by one or more of the binding molecules disclosed. Therefore, for example, certain binding molecules described herein can bind to Psl-like and PcrV-like molecules produced by other bacterial species, for example, Psl-like or PcrV-like molecules produced by other Pseudomonas species, e.g., Pseudomonas fluorescens, Pseudomonas putida, or Pseudomonas alcaligenes. Alternatively, certain binding molecules as described herein can bind to Psl-like and PcrV-like molecules produced synthetically or by host cells genetically modified to produce Psl-like or PcrV-like molecules.

Unless it is specifically noted, as used herein a “fragment thereof” in reference to a binding molecule, e.g., an antibody refers to an antigen-binding fragment, i.e., a portion of the antibody that specifically binds to the antigen.

Anti-Pseudomonas Psl and PcrV bispecific binding molecules, e.g., bispecific antibodies or antigen-binding fragments, variants, or derivatives thereof can comprise a constant region that mediates one or more effector functions. For example, binding of the C1 component of complement to an antibody constant region can activate the complement system. Activation of complement is important in the opsonization and lysis of pathogens. The activation of complement also stimulates the inflammatory response and can also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments disclosed herein include an anti-Pseudomonas Psl and PcrV bispecific binding molecule, e.g., a bispecific antibody, or antigen-binding fragment, variant, or derivative thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain binding molecules described herein are domain deleted antibodies that comprise a polypeptide chain similar to an immunoglobulin heavy chain, but lack at least a portion of one or more heavy chain domains. For instance, in certain antibodies, one entire domain of the constant region of the modified antibody will be deleted, for example, all or part of the CH2 domain will be deleted.

Modified forms of anti-Pseudomonas Psi and PcrV bispecific binding molecules, e.g., bispecific antibodies or antigen-binding fragments, variants, or derivatives thereof can be made from whole precursor or parent antibodies using techniques known in the art. Exemplary techniques are discussed elsewhere herein.

In certain embodiments both the variable and constant regions of anti-Pseudomonas Psi and PcrV bispecific binding molecules, e.g., bispecific antibodies or antigen-binding fragments are fully human. Fully human antibodies can be made using techniques that are known in the art and as described herein. For example, fully human antibodies against a specific antigen can be prepared by administering the antigen to a transgenic animal that has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human anti bodies can likewise be produced by various display technologies, e.g., phage display or other viral display systems, as described in more detail elsewhere herein.

Anti-Pseudomonas Psi and PcrV bispecific binding molecules, e.g., bispecific antibodies or antigen-binding fragments, variants, or derivatives thereof as disclosed herein can be made or manufactured using techniques that are known in the art. In certain embodiments, binding molecules or fragments thereof are “recombinantly produced,” i.e., are produced using recombinant DNA technology. Exemplary techniques for making antibody molecules or fragments thereof are discussed in more detail elsewhere herein.

In certain anti-Pseudomonas Psi and PcrV bispecific binding molecules, e.g., bispecific antibodies or antigen-binding fragments, variants, or derivatives thereof described herein, the Fc portion can be mutated to decrease effector function using techniques known in the art. For example, the deletion or inactivation (through point mutations or other means) of a constant region domain can reduce Fc receptor binding of the circulating modified antibody thereby increasing tumor localization. In other cases it can be that constant region modifications moderate complement binding and thus reduce the serum half-life and nonspecific association of a conjugated cytotoxin. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as localization, biodistribution and serum half-life, can easily be measured and quantified using well known immunological techniques without undue experimentation.

In certain embodiments, anti-Pseudomonas Psl and PcrV bispecific binding molecules, e.g., bispecific antibodies or antigen-binding fragments, variants, or derivatives thereof will not elicit a deleterious immune response in the animal to be treated, e.g., in a human. In one embodiment, anti-Pseudomonas Psl and PcrV bispecific binding molecules, e.g., bispecific antibodies or antigen-binding fragments, variants, or derivatives thereof are modified to reduce their immunogenicity using art-recognized techniques. For example, antibodies can be humanized, de-immunized, or chimeric antibodies can be made. These types of antibodies are derived from a non-human antibody, typically a murine or primate antibody, that retains or substantially retains the antigen-binding properties of the parent antibody, but is less immunogenic in humans. This can be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., Pseudomonas Psl- and PcrV-bispecific antibodies or antigen-binding fragments thereof disclosed herein, which are then tested for function. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

Anti-Pseudomonas Psl and PcrV bispecific binding molecules, e.g., bispecific antibodies or antigen-binding fragments, variants, or derivatives thereof can be generated by any suitable method known in the art. Polyclonal antibodies to an antigen of interest can be produced by various procedures well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988)

DNA encoding antibodies or antibody fragments (e.g., antigen binding sites) can also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with scFv, Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles that carry the polynucleotide sequences encoding them. For example, DNA sequences encoding VH and VL regions are amplified from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., Pseudomonas Psl or PcrV) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references and in the examples below, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques that can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). In certain embodiments such as therapeutic administration, chimeric, humanized, or human antibodies can be used. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Fully human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice that are incapable of expressing functional endogenous immunoglobulins, but can express human immunoglobulin genes. For example, the human heavy and light chain immunoglobulin gene complexes can be introduced randomly or by homologous recombination into mouse embryonic stem cells. In addition, various companies can be engaged to provide human antibodies produced in transgenic mice directed against a selected antigen using technology similar to that described above.

Fully human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

In another embodiment, DNA encoding desired monoclonal antibodies can be readily isolated and sequenced using conventional procedures (e.g., by using oligonucleotide probes that are capable of binding specifically to genes encoding the heavy and light chains of murine antibodies). Isolated and subcloned hybridoma cells or isolated phage, for example, can serve as a source of such DNA. Once isolated, the DNA can be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which can be synthetic as described herein) can be used to clone constant and variable region sequences for the manufacture antibodies as described in Newman et al., U.S. Pat. No. 5,658,570, filed Jan. 25, 1995, which is incorporated by reference herein. Transformed cells expressing the desired antibody can be grown up in relatively large quantities to provide clinical and commercial supplies of the immunoglobulin.

In one embodiment, an isolated binding molecule, e.g., an antibody comprises at least one heavy or light chain CDR of an antibody molecule. In another embodiment, an isolated binding molecule comprises at least two CDRs from one or more antibody molecules. In another embodiment, an isolated binding molecule comprises at least three CDRs from one or more antibody molecules. In another embodiment, an isolated binding molecule comprises at least four CDRs from one or more antibody molecules. In another embodiment, an isolated binding molecule comprises at least five CDRs from one or more antibody molecules. In another embodiment, an isolated binding molecule of the description comprises at least six CDRs from one or more antibody molecules.

In a specific embodiment, the amino acid sequence of the heavy and/or light chain variable domains can be inspected to identify the sequences of the complementarity determining regions (CDRs) by methods that are well-known in the art, e.g., by comparison to known amino acid sequences of other heavy and light chain variable regions to determine the regions of sequence hypervariability. Using routine recombinant DNA techniques, one or more of the CDRs can be inserted within framework regions, e.g., into human framework regions to humanize a non-human antibody. The framework regions can be naturally occurring or consensus framework regions, and preferably human framework regions (see, e.g., Chothia et al., J. Mol. Biol. 278:457-479 (1998) for a listing of human framework regions). The polynucleotide generated by the combination of the framework regions and CDRs encodes an antibody that specifically binds to at least one epitope of a desired antigen, e.g., Psl or PcrV. One or more amino acid substitutions can be made within the framework regions, and, the amino acid substitutions improve binding of the antibody to its antigen. Additionally, such methods can be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present disclosure and are within the capabilities of a person of skill of the art.

Also provided are bispecific binding molecules that comprise, consist essentially of, or consist of, variants (including derivatives) of antibody molecules (e.g., the VH regions and/or VL regions) described herein, which binding molecules or fragments thereof specifically bind to Pseudomonas Psl and PcrV. Standard techniques known to those of skill in the art can be used to introduce mutations in the nucleotide sequence encoding a binding molecule or fragment thereof that specifically binds to Pseudomonas Psl and PcrV, including, but not limited to, site-directed mutagenesis and PCR-mediated mutagenesis that results in amino acid substitutions. The variants (including derivatives) encode polypeptides comprising less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an Pseudomonas Psl and PcrV).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to bind at least one epitope of Pseudomonas Psl and PcrV) can be determined using techniques described herein or by routinely modifying techniques known in the art.

One embodiment provides a bispecific antibody comprising anti-Pseudomonas Psl and PcrV binding domains disclosed herein. In certain embodiments, the bispecific antibody contains a first Psl binding domain, and the second PcrV binding domain. Bispecific antibodies with more than two valencies are contemplated. For example, trispecific antibodies can also be prepared using the methods described herein. (Tutt et al., J. Immunol., 147:60 (1991)).

One embodiment provides a method of producing a bispecific antibody, that utilizes a single light chain that can pair with both heavy chain variable domains present in the bispecific molecule. To identify this light chain, various strategies can be employed. In one embodiment, a series of monoclonal antibodies are identified to each antigen that can be targeted with the bispecific antibody, followed by a determination of which of the light chains utilized in these antibodies is able to function when paired with the heavy chain of any of the antibodies identified to the second target. In this manner a light chain that can function with two heavy chains to enable binding to both antigens can be identified. In another embodiment, display techniques, such as phage display, can enable the identification of a light chain that can function with two or more heavy chains. In one embodiment, a phage library is constructed that comprises a diverse repertoire of heavy chain variable domains and a single light chain variable domain. This library can further be utilized to identify antibodies that bind to various antigens of interest. Thus, in certain embodiments, the antibodies identified will share a common light chain.

In certain embodiments, the bispecific antibody comprises at least one single chain Fv (scFv). In certain embodiments the bispecific antibody comprises two scFvs. For example, a scFv can be fused to one or both of a CH3 domain-containing polypeptide contained within an antibody. Some methods comprise producing a bispecific molecule wherein one or both of the heavy chain constant regions comprising at least a CH3 domain is utilized in conjunction with a single chain Fv domain to provide antigen binding.

### III. Antibody Polypeptides

The disclosure is further directed to isolated polypeptides that make up binding molecules, e.g., bispecific antibodies or antigen-binding fragments thereof, which specifically bind to Pseudomonas Psl and PcrV and polynucleotides encoding such polypeptides. Binding molecules, e.g., bispecific antibodies or fragments thereof as disclosed herein, comprise polypeptides, e.g., amino acid sequences encoding, for example, Psl-specific and PcrV-specific antigen binding regions derived from immunoglobulin molecules. A polypeptide or amino acid sequence “derived from” a designated protein refers to the origin of the polypeptide. In certain cases, the polypeptide or amino acid sequence that is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or that is otherwise identifiable to one of ordinary skill in the art as having its origin in the starting sequence.

A bispecific binding molecule as provided herein can include an antibody single chain Fv (ScFv) fragment that specifically binds to Pseudomonas Psl (an “anti-Psl ScFv”), comprising the formula VH-L-VL or alternatively VL-L-VH, where L is a linker sequence. In certain aspects the linker can comprise (a) [GGGGS]n, wherein n is 0, 1, 2, 3, 4, or 5 (SEQ ID NO: 2), (b) [GGGG]n, wherein n is 0, 1, 2, 3, 4, or 5 (SEQ ID NO: 3), or a combination of (a) and (b). For example, an exemplary linker comprises: GGGGSGGGGSGGGGSGGGGSGGGGS (SEQ ID NO: 4). In certain embodiments the linker further comprises the amino acids ala-leu at the C-terminus of the linker.

In certain aspects the anti-Psl ScFv comprises the VH and VL of Psl0096. In certain aspects the anti-Psl ScFv comprises the amino acid sequence:

(SEQ ID NO: 5)
QVQLQESGPGLVKPSETLSLTCTVSGGSISPYYWTWIRQPPGKCLELIGY
IHSSGYTDYNPSLKSRVTISGDTSKKQFSLKLSSVTAADTAVYYCARADW
DRLRALDIWGQGTMVTVSSGGGGSGGGGSGGGGSGGGGSDIQLTQSPSSL
SASVGDRVTITCRASQSIRSHLNWYQQKPGKAPKLLIYGASNLQSGVPSR
FSGSGSGTDFTLTISSLQPEDFATYYCQQSTGAWNWFGCGTKVEIK.

Also disclosed is a bispecific binding molecule comprising a single chain Fv (ScFv) fragment that specifically binds to Pseudomonas PcrV (an “anti-PcrV ScFv”), comprising the formula VH-L-VL or alternatively VL-L-VH, where L is a linker sequence. In certain aspects the linker can comprise (a) [GGGGS]n, wherein n is 0, 1, 2, 3, 4, or 5 (SEQ ID NO: 2), (b) [GGGG]n, wherein n is 0, 1, 2, 3, 4, or 5 (SEQ ID NO: 3), or a combination of (a) and (b). For example, an exemplary linker comprises: GGGGSGGGGSGGGGSGGGGSGGGGS (SEQ ID NO: 4). In certain embodiments the linker further comprises the amino acids ala-leu at the C-terminus of the linker.

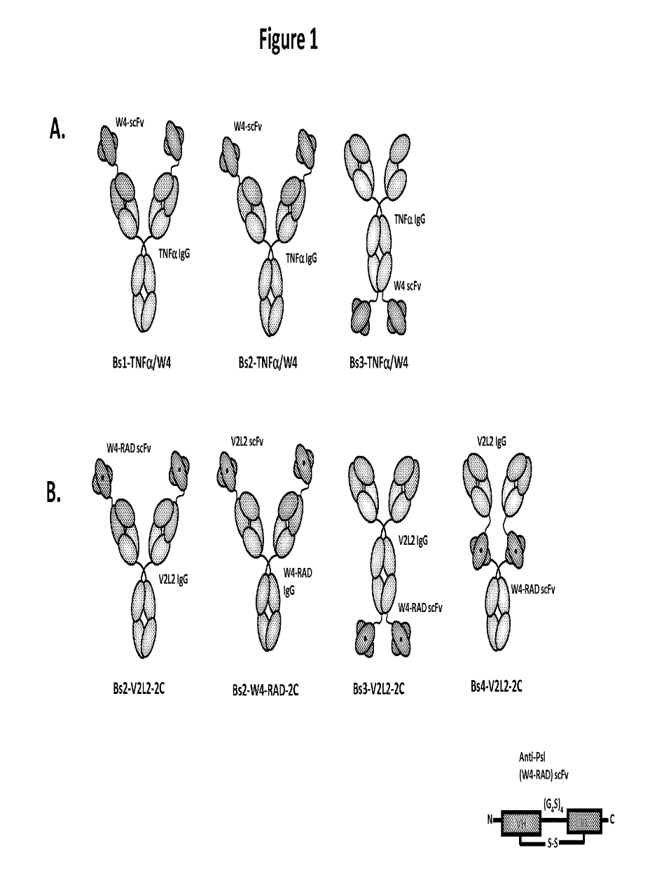

In certain embodiments, a bispecific antibody as disclosed herein has the structure of BS2, BS3, or BS4, all as shown in FIG. 1. In certain bispecific antibodies disclosed herein the binding domain that specifically binds to Pseudomonas Psl comprises an anti-Psl ScFv molecule. In other aspects the binding domain that specifically binds to Pseudomonas Psl comprises a conventional heavy chain and light chain. Similarly in certain bispecific antibodies disclosed herein the binding domain that specifically binds to Pseudomonas PcrV comprises an anti-PcrV ScFv molecule. In other aspects the binding domain that specifically binds to Pseudomonas PcrV comprises a conventional heavy chain and light chain.

The structures used for the bispecific antibodies disclosed herein are described detail in U.S. Provisional Appl. No. 61/624,651 filed on Apr. 16, 2012 and International Application No: PCT/US2012/63639, filed Nov. 6, 2012, published as WO2013/070565 on May 16, 2013 (attorney docket no. AEMS-115WO1, entitled “MULTISPECIFIC AND MULTIVALENT BINDING PROTEINS AND USES THEREOF”), which is incorporated herein by reference in its entirety.

This disclosure provides a bispecific antibody that specifically binds to Pseudomonas Psl and PcrV in the Bs2 format, comprising an antibody heavy chain and an antibody light chain, where the antibody heavy chain comprises the formula S-VH-CH1-Hi-CH2-CH3, where S is an anti Psl ScFv molecule, VH is an anti-PcrV heavy chain variable domain, CH1 is a heavy chain constant region domain-1, e.g., a human heavy chain constant region domain-1, Hi is the heavy chain hinge region, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the heavy chain comprises the amino acid sequence:

Bs2-V2L2MD/Ps10096 Amino acid-HC:
(SEQ ID NO: 6)
QVQLQESGPGLVKPSETLSLTCTVSGGSISPYYWTWIRQPPGKCLELIGY
IHSSGYTDYNPSLKSRVTISGDTSKKQFSLKLSSVTAADTAVYYCARADW
DRLRALDIWGQGTMVTVSSGGGGSGGGGSGGGGSGGGGSDIQLTQSPSSL
SASVGDRVTITCRASQSIRSHLNWYQQKPGKAPKLLIYGASNLQSGVPSR
FSGSGSGTDFTLTISSLQPEDFATYYCQQSTGAWNWFGCGTKVEIKGGGG
SGGGGSEVQLLESGGGLVQPGGSLRLSCAASGFTFSSYAMNWVRQAPGKG
LEWVSAITMSGITAYYTDDVKGRFTISRDNSKNTLYLQMNSLRAEDTAVY
YCAKEEFLPGTHYYYGMDVWGQGTTVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYT
QKSLSLSPGK;

and in certain aspects the light chain comprises the amino acid sequence:

Bs2-V2L2MD/Ps10096 Amino acid-LC:
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

This disclosure further provides a bispecific antibody that specifically binds to Pseudomonas Psl and PcrV in the Bs3 format, comprising an antibody heavy chain and an antibody light chain, where the antibody heavy chain comprises the formula VH-CH1-Hi-CH2-CH3-S, where S is an anti Psl ScFv molecule, VH is an anti-PcrV heavy chain variable domain, CH1 is a heavy chain constant region domain-1, e.g., a human heavy chain constant region domain-1, Hi is the heavy chain hinge region, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the heavy chain comprises the amino acid sequence:

Bs3-V2L2MD/Ps10096 Amino acid - HC
(SEQ ID NO: 8)
EVQLLESGGGLVQPGGSLRLSCAASGFTFSSYAMNWVRQAPGKGLEWVSA
ITMSGITAYYTDDVKGRFTISRDNSKNTLYLQMNSLRAEDTAVYYCAKEE
FLPGTHYYYGMDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGKGGGGSGGGGSQVQLQESGPGLVKPSETLSLTCTVSGGSISPYYWTW
IRQPPGKCLELIGYIHSSGYTDYNPSLKSRVTISGDTSKKQFSLKLSSVT
AADTAVYYCARADWDRLRALDIWGQGTMVTVSSGGGGSGGGGSGGGGSGG
GGSDIQLTQSPSSLSASVGDRVTITCRASQSIRSHLNWYQQKPGKAPKLL
IYGASNLQSGVPSRFSGSGSGTDFTLTISSLQPEDFATYYCQQSTGAWNW
FGCGTKVEIK;

and the light chain comprises the amino acid sequence

Bs3-V2L2MD/Ps10096 Amino acid - LC
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

This disclosure provides a bispecific antibody that specifically binds to Pseudomonas Psl and PcrV in the Bs4 format, comprising an antibody heavy chain and an antibody light chain, where the heavy chain comprises the formula VH-CH1-H1-L1-S-L2-H2-CH2-CH3, where VH is an anti-PcrV heavy chain variable region, CH1 is a heavy chain constant region domain-1, e.g., a human heavy chain constant region domain-1, H1 is a first heavy chain hinge region fragment, L1 is a first linker, S is an anti-Psl ScFv molecule, L2 is a second linker, H2 is a second heavy chain hinge region fragment, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the heavy chain comprises the amino acid sequence:

Bs4-V2L2MD/Ps10096 Amino acid - HC
(SEQ ID NO: 9)
EVQLLESGGGLVQPGGSLRLSCAASGFTFSSYAMNWVRQAPGKGLEWVSA
ITMSGITAYYTDDVKGRFTISRDNSKNTLYLQMNSLRAEDTAVYYCAKEE
FLPGTHYYYGMDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCGGGGSGGGGSQVQLQESGPGLVK
PSETLSLTCTVSGGSISPYYWTWIRQPPGKCLELIGYIHSSGYTDYNPSL
KSRVTISGDTSKKQFSLKLSSVTAADTAVYYCARADWDRLRALDIWGQGT
MVTVSSGGGGSGGGGSGGGGSGGGGSDIQLTQSPSSLSASVGDRVTITCR
ASQSIRSHLNWYQQKPGKAPKLLIYGASNLQSGVPSRFSGSGSGTDFTLT
ISSLQPEDFATYYCQQSTGAWNWFGCGTKVEIKGGGGSGGGGSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK;

and the light chain comprises the amino acid sequence

Bs4-V2L2MD/Ps10096 Amino acid - LC
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

An anti-Pseudomonas Psl and PcrV bispecific binding molecule, e.g., an antibody or fragment, variant or derivative thereof described herein can comprise, consist essentially of, or consist of a fusion protein. Fusion proteins are chimeric molecules that comprise, for example, an immunoglobulin antigen-binding domain with at least one target binding site, and at least one heterologous portion, i.e., a portion with which it is not naturally linked in nature. The amino acid sequences can normally exist in separate proteins that are brought together in the fusion polypeptide or they can normally exist in the same protein but are placed in a new arrangement in the fusion polypeptide. Fusion proteins can be created, for example, by chemical synthesis, or by creating and translating a polynucleotide in which the peptide regions are encoded in the desired relationship.

The term “heterologous” as applied to a polynucleotide, polypeptide, or other moiety means that the polynucleotide, polypeptide, or other moiety is derived from a distinct entity from that of the rest of the entity to which it is being compared. In a non-limiting example, a “heterologous polypeptide” to be fused to a binding molecule, e.g., an antibody or an antigen-binding fragment, variant, or derivative thereof is derived from a non-immunoglobulin polypeptide of the same species, or an immunoglobulin or non-immunoglobulin polypeptide of a different species.

### IV. Fusion Proteins and Antibody Conjugates

In some embodiments, the anti-Pseudomonas Psl and PcrV bispecific binding molecules, e.g., bispecific antibodies or fragments, variants or derivatives thereof can be administered multiple times in conjugated form. In still another embodiment, the anti-Pseudomonas Psl and PcrV bispecific binding molecules, e.g., bispecific antibodies or fragments, variants or derivatives thereof can be administered in unconjugated form, then in conjugated form, or vice versa.

In specific embodiments, anti-Pseudomonas Psi and PcrV bispecific binding molecules, e.g., bispecific antibodies or fragments, variants or derivatives thereof can be conjugated to one or more antimicrobial agents, for example, Polymyxin B (PMB). PMB is a small lipopeptide antibiotic approved for treatment of multidrug-resistant Gram-negative infections. In addition to its bactericidal activity, PMB binds lipopolysaccharide (LPS) and neutralizes its proinflammatory effects. (Dixon, R. A. & Chopra, I. J Antimicrob Chemother 18, 557-563 (1986)). LPS is thought to significantly contribute to inflammation and the onset of Gram-negative sepsis. (Guidet, B., et al., Chest 106, 1194-1201 (1994)). Conjugates of PMB to carrier molecules have been shown to neutralize LPS and mediate protection in animal models of endotoxemia and infection. (Drabick, J. J., et al. Antimicrob Agents Chemother 42, 583-588 (1998)). Also disclosed is a method for attaching one or more PMB molecules to cysteine residues introduced into the Fc region of monoclonal antibodies (mAb) of the disclosure. For example, the Cam-003-PMB conjugates retained specific, mAb-mediated binding to P. aeruginosa and also retained OPK activity. Furthermore, mAb-PMB conjugates bound and neutralized LPS in vitro. In certain embodiments, anti-Pseudomonas Psi and PcrV bispecific binding molecules, e.g., bispecific antibodies or fragments, variants or derivatives thereof can be combined with antibiotics (e.g., Ciprofloxacin, Meropenem, Tobramycin, Aztreonam).

In certain embodiments, an anti-Pseudomonas Psi and PcrV bispecific binding molecule, e.g., an antibody or fragment, variant or derivative thereof described herein can comprise a heterologous amino acid sequence or one or more other moieties not normally associated with an antibody (e.g., an antimicrobial agent, a therapeutic agent, a prodrug, a peptide, a protein, an enzyme, a lipid, a biological response modifier, pharmaceutical agent, a lymphokine, a heterologous antibody or fragment thereof, a detectable label, polyethylene glycol (PEG), and a combination of two or more of any said agents). In further embodiments, an anti-Pseudomonas Psi and PcrV bispecific binding molecule, e.g., an antibody or fragment, variant or derivative thereof can comprise a detectable label selected from the group consisting of an enzyme, a fluorescent label, a chemiluminescent label, a bioluminescent label, a radioactive label, or a combination of two or more of any said detectable labels.

### V. Polynucleotides Encoding Binding Molecules

Also provided herein are nucleic acid molecules encoding the anti-Pseudomonas Psl and PcrV bispecific binding molecules, e.g., bispecific antibodies or fragments, variants or derivatives thereof described herein.

One embodiment provides one or more isolated polynucleotides comprising, consisting essentially of, or consisting of nucleic acids that encodes a bispecific antibody that specifically binds to Pseudomonas Psl and PcrV in the Bs2 format, comprising a nucleic acid that encodes an antibody heavy chain and a nucleic acid that encodes an antibody light chain, where the antibody heavy chain comprises the formula S-VH-CH1-Hi-CH2-CH3, where S is an anti Psl ScFv molecule, VH is an anti-PcrV heavy chain variable domain, CH1 is a heavy chain constant region domain-1, e.g., a human heavy chain constant region domain-1, Hi is the heavy chain hinge region, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the polynucleotide encoding the heavy chain comprises the nucleic acid sequence:

Bs2-V2L2MD/Ps10096 Nucleotide - HC
(SEQ ID NO: 10)
CAGGTGCAGCTGCAGGAATCTGGCCCTGGCCTCGTGAAGCCCTCCGAGAC
ACTGTCTCTGACCTGCACCGTGTCCGGCGGCTCCATCTCCCCTTACTACT
GGACCTGGATCAGACAGCCCCCTGGCAAGTGCCTGGAACTGATCGGCTAC
ATCCACTCCTCCGGCTACACCGACTACAACCCCAGCCTGAAGTCCAGAGT
GACCATCTCCGGCGACACCTCCAAGAAGCAGTTCTCCCTGAAGCTGTCCT
CCGTGACCGCCGCTGATACCGCCGTGTACTACTGCGCCAGAGCCGACTGG
GACAGACTGAGAGCCCTGGACATCTGGGGCCAGGGCACAATGGTCACCGT
GTCTAGCGGAGGCGGAGGATCTGGTGGTGGTGGATCTGGCGGCGGAGGAA
GTGGTGGCGGAGGCTCTGATATCCAGCTGACCCAGTCCCCCTCCAGCCTG
TCTGCTTCTGTGGGCGACCGCGTGACCATCACCTGTAGAGCCTCCCAGTC
CATCCGGTCCCACCTGAACTGGTATCAGCAGAAGCCCGGCAAGGCCCCCA
AGCTGCTGATCTACGGCGCCTCCAATCTGCAGTCCGGCGTGCCCTCTAGA
TTCTCCGGATCTGGCTCCGGCACCGACTTTACCCTGACCATCAGCTCCCT
GCAGCCCGAGGACTTCGCCACCTACTACTGCCAGCAGTCTACCGGCGCCT
GGAATTGGTTCGGCTGCGGCACCAAGGTGGAAATCAAGGGCGGAGGGGGA
TCCGGCGGAGGGGGCTCTGAGGTGCAGCTGTTGGAGTCTGGGGGAGGCTT
GGTACAGCCTGGGGGGTCCCTGAGACTCTCCTGTGCAGCCTCTGGATTCA
CCTTTAGCAGCTATGCCATGAACTGGGTCCGCCAGGCTCCAGGGAAGGGG
CTGGAGTGGGTCTCAGCTATTACTATGAGTGGTATTACCGCATACTACAC
CGACGACGTGAAGGGCCGGTTCACCATCTCCAGAGACAATTCCAAGAACA
CGCTATATCTGCAAATGAACAGCCTGAGGGCCGAGGACACGGCCGTATAT
TACTGTGCGAAGGAAGAATTTTTACCTGGAACGCACTACTACTACGGTAT
GGACGTCTGGGGCCAAGGGACCACGGTCACCGTCTCCTCAGCGTCGACCA
AGGGCCCATCCGTCTTCCCCCTGGCACCCTCCTCCAAGAGCACCTCTGGG
GGCACAGCGGCCCTGGGCTGCCTGGTCAAGGACTACTTCCCCGAACCGGT
GACGGTGTCCTGGAACTCAGGCGCTCTGACCAGCGGCGTGCACACCTTCC
CGGCTGTCCTACAGTCCTCAGGACTCTACTCCCTCAGCAGCGTGGTGACC
GTGCCCTCCAGCAGCTTGGGCACCCAGACCTACATCTGCAACGTGAATCA
CAAGCCCAGCAACACCAAGGTGGACAAGAGAGTTGAGCCCAAATCTTGTG
ACAAAACTCACACATGCCCACCGTGCCCAGCACCTGAACTCCTGGGGGGA
CCGTCAGTCTTCCTCTTCCCCCCAAAACCCAAGGACACCCTCATGATCTC
CCGGACCCCTGAGGTCACATGCGTGGTGGTGGACGTGAGCCACGAAGACC
CTGAGGTCAAGTTCAACTGGTACGTGGACGGCGTGGAGGTGCATAATGCC
AAGACAAAGCCGCGGGAGGAGCAGTACAACAGCACGTACCGTGTGGTCAG
CGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCC
AAAGCCAAAGGGCAGCCCCGAGAACCACAGGTCTACACCCTGCCCCCATC
CCGGGAGGAGATGACCAAGAACCAGGTCAGCCTGACCTGCCTGGTCAAAG
GCTTCTATCCCAGCGACATCGCCGTGGAGTGGGAGAGCAATGGGCAGCCG
GAGAACAACTACAAGACCACGCCTCCCGTGCTGGACTCCGACGGCTCCTT
CTTCCTCTATAGCAAGCTCACCGTGGACAAGAGCAGGTGGCAGCAGGGGA
ACGTCTTCTCATGCTCCGTGATGCATGAGGCTCTGCACAACCACTACACG
CAGAAGAGCTTAAGCCTGTCTCCGGGTAAA;

and in certain aspects the polynucleotide encoding the light chain comprises the nucleic acid sequence:

Bs2-V2L2MD/Ps10096 Nucleotide - LC:
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT.

One embodiment provides one or more isolated polynucleotides comprising, consisting essentially of, or consisting of nucleic acids that encodes a bispecific antibody that specifically binds to Pseudomonas Psl and PcrV in the Bs3 format, comprising a nucleic acid that encodes an antibody heavy chain and a nucleic acid that encodes an antibody light chain, where the antibody heavy chain comprises the formula VH-CH1-Hi-CH2-CH3-S, where S is an anti Psl ScFv molecule, VH is an anti-PcrV heavy chain variable domain, CH1 is a heavy chain constant region domain-1, e.g., a human heavy chain constant region domain-1, Hi is the heavy chain hinge region, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the polynucleotide encoding the heavy chain comprises the nucleic acid sequence:

Bs3-V2L2MD/Psl0096 Nucleotide-HC
(SEQ ID NO: 12)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAACTGGGTCCGCCAGGCTCCAGGGAAGGGGCTGGAGTGGGTCTCAGCT
ATTACTATGAGTGGTATTACCGCATACTACACCGACGACGTGAAGGGCCG
GTTCACCATCTCCAGAGACAATTCCAAGAACACGCTATATCTGCAAATGA
ACAGCCTGAGGGCCGAGGACACGGCCGTATATTACTGTGCGAAGGAAGAA
TTTTTACCTGGAACGCACTACTACTACGGTATGGACGTCTGGGGCCAAGG
GACCACGGTCACCGTCTCCTCAGCGTCGACCAAGGGCCCATCCGTCTTCC
CCCTGGCACCCTCCTCCAAGAGCACCTCTGGGGGCACAGCGGCCCTGGGC
TGCCTGGTCAAGGACTACTTCCCCGAACCGGTGACGGTGTCCTGGAACTC
AGGCGCTCTGACCAGCGGCGTGCACACCTTCCCGGCTGTCCTACAGTCCT
CAGGACTCTACTCCCTCAGCAGCGTGGTGACCGTGCCCTCCAGCAGCTTG
GGCACCCAGACCTACATCTGCAACGTGAATCACAAGCCCAGCAACACCAA
GGTGGACAAGAGAGTTGAGCCCAAATCTTGTGACAAAACTCACACATGCC
CACCGTGCCCAGCACCTGAACTCCTGGGGGGACCGTCAGTCTTCCTCTTC
CCCCCAAAACCCAAGGACACCCTCATGATCTCCCGGACCCCTGAGGTCAC
ATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAG
GAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCC
CGAGAACCACAGGTCTACACCCTGCCCCCATCCCGGGAGGAGATGACCAA
GAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCG
TGATGCATGAGGCTCTGCACAACCACTACACGCAGAAGAGCTTAAGCCTG
TCTCCGGGTAAAGGCGGAGGGGGATCCGGCGGAGGGGGCTCTCAGGTGCA
GCTGCAGGAATCTGGCCCTGGCCTCGTGAAGCCCTCCGAGACACTGTCTC
TGACCTGCACCGTGTCCGGCGGCTCCATCTCCCCTTACTACTGGACCTGG
ATCAGACAGCCCCCTGGCAAGTGCCTGGAACTGATCGGCTACATCCACTC
CTCCGGCTACACCGACTACAACCCCAGCCTGAAGTCCAGAGTGACCATCT
CCGGCGACACCTCCAAGAAGCAGTTCTCCCTGAAGCTGTCCTCCGTGACC
GCCGCTGATACCGCCGTGTACTACTGCGCCAGAGCCGACTGGGACAGACT
GAGAGCCCTGGACATCTGGGGCCAGGGCACAATGGTCACCGTGTCTAGCG
GAGGCGGAGGATCTGGTGGTGGTGGATCTGGCGGCGGAGGAAGTGGTGGC
GGAGGCTCTGATATCCAGCTGACCCAGTCCCCCTCCAGCCTGTCTGCTTC
TGTGGGCGACCGCGTGACCATCACCTGTAGAGCCTCCCAGTCCATCCGGT
CCCACCTGAACTGGTATCAGCAGAAGCCCGGCAAGGCCCCCAAGCTGCTG
ATCTACGGCGCCTCCAATCTGCAGTCCGGCGTGCCCTCTAGATTCTCCGG
ATCTGGCTCCGGCACCGACTTTACCCTGACCATCAGCTCCCTGCAGCCCG
AGGACTTCGCCACCTACTACTGCCAGCAGTCTACCGGCGCCTGGAATTGG
TTCGGCTGCGGCACCAAGGTGGAAATCAAG;

and the polynucleotide encoding the light chain comprises the nucleic acid sequence

Bs3-V2L2MD/Psl0096 Nucleotide-LC
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT.

One embodiment provides one or more isolated polynucleotides comprising, consisting essentially of, or consisting of nucleic acids that encodes a bispecific antibody that specifically binds to Pseudomonas Psl and PcrV in the Bs4 format, comprising a nucleic acid that encodes an antibody heavy chain and a nucleic acid that encodes an antibody light chain, where the heavy chain comprises the formula VH-CH1-H1-L1-S-L2-H2-CH2-CH3, where VH is an anti-PcrV heavy chain variable region, CH1 is a heavy chain constant region domain-1, e.g., a human heavy chain constant region domain-1, H1 is a first heavy chain hinge region fragment, L1 is a first linker, S is an anti-Psl ScFv molecule, L2 is a second linker, H2 is a second heavy chain hinge region fragment, CH2 is a heavy chain constant region domain-2, e.g., a human heavy chain constant region domain-2, and CH3 is a heavy chain constant region domain-3, e.g., a human heavy chain constant region domain-3, and where the light chain comprises VL-CL, where VL is an anti-PcrV light chain variable domain and CL is a light chain constant region, e.g., a human kappa light chain constant region. In certain aspects the polynucleotide encoding the heavy chain comprises the nucleic acid sequence:

Bs4-V2L2MD/Psl0096 Nucleotide-HC
(SEQ ID NO: 13)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAACTGGGTCCGCCAGGCTCCAGGGAAGGGGCTGGAGTGGGTCTCAGCT
ATTACTATGAGTGGTATTACCGCATACTACACCGACGACGTGAAGGGCCG
GTTCACCATCTCCAGAGACAATTCCAAGAACACGCTATATCTGCAAATGA
ACAGCCTGAGGGCCGAGGACACGGCCGTATATTACTGTGCGAAGGAAGAA
TTTTTACCTGGAACGCACTACTACTACGGTATGGACGTCTGGGGCCAAGG
GACCACGGTCACCGTCTCCTCAGCGTCGACCAAGGGCCCATCCGTCTTCC
CCCTGGCACCCTCCTCCAAGAGCACCTCTGGGGGCACAGCGGCCCTGGGC
TGCCTGGTCAAGGACTACTTCCCCGAACCGGTGACGGTGTCCTGGAACTC
AGGCGCTCTGACCAGCGGCGTGCACACCTTCCCGGCTGTCCTACAGTCCT
CAGGACTCTACTCCCTCAGCAGCGTGGTGACCGTGCCCTCCAGCAGCTTG
GGCACCCAGACCTACATCTGCAACGTGAATCACAAGCCCAGCAACACCAA
GGTGGACAAGAGAGTTGAGCCCAAATCTTGTGGCGGAGGGGGCTCTGGCG
GAGGGGGATCCCAGGTGCAGCTGCAGGAATCTGGCCCTGGCCTCGTGAAG
CCCTCCGAGACACTGTCTCTGACCTGCACCGTGTCCGGCGGCTCCATCTC
CCCTTACTACTGGACCTGGATCAGACAGCCCCCTGGCAAGTGCCTGGAAC
TGATCGGCTACATCCACTCCTCCGGCTACACCGACTACAACCCCAGCCTG
AAGTCCAGAGTGACCATCTCCGGCGACACCTCCAAGAAGCAGTTCTCCCT
GAAGCTGTCCTCCGTGACCGCCGCTGATACCGCCGTGTACTACTGCGCCA
GAGCCGACTGGGACAGACTGAGAGCCCTGGACATCTGGGGCCAGGGCACA
ATGGTCACCGTGTCTAGCGGAGGCGGAGGATCTGGTGGTGGTGGATCTGG
CGGCGGAGGAAGTGGTGGCGGAGGCTCTGATATCCAGCTGACCCAGTCCC
CCTCCAGCCTGTCTGCTTCTGTGGGCGACCGCGTGACCATCACCTGTAGA
GCCTCCCAGTCCATCCGGTCCCACCTGAACTGGTATCAGCAGAAGCCCGG
CAAGGCCCCCAAGCTGCTGATCTACGGCGCCTCCAATCTGCAGTCCGGCG
TGCCCTCTAGATTCTCCGGATCTGGCTCCGGCACCGACTTTACCCTGACC
ATCAGCTCCCTGCAGCCCGAGGACTTCGCCACCTACTACTGCCAGCAGTC
TACCGGCGCCTGGAATTGGTTCGGCTGCGGCACCAAGGTGGAAATCAAGG
GCGGAGGTGGCTCTGGCGGAGGGGGATCCGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTCTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCTTAAGCCTGTC
TCCGGGTAAA;

and the polynucleotide encoding the light chain comprises the nucleic acid sequence

Bs4-V2L2MD/Psl0096 Nucleotide-LC
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT.

In other embodiments, an anti-Pseudomonas Psl and PcrV bispecific binding molecule, e.g., antibody or fragment, variant or derivative thereof encoded by one or more of the polynucleotides described above, specifically binds to the same epitope as monoclonal antibody WapR-004, W4-RAD, or W4-RAD-2C, or will competitively inhibit such a monoclonal antibody from binding to Pseudomonas Psl.

The disclosure also includes fragments of the polynucleotides as described elsewhere herein. Additionally polynucleotides that encode fusion polynucleotides, Fab fragments, and other derivatives, as described herein, are also provided.

The polynucleotides can be produced or manufactured by any method known in the art. For example, if the nucleotide sequence of the antibody is known, a polynucleotide encoding the antibody can be assembled from chemically synthesized oligonucleotides (e.g., as described in Kutmeier et al., BioTechniques 17:242 (1994)), that, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an anti-Pseudomonas Psl and PcrV bispecific binding molecule, e.g., a bispecific antibody or fragment, variant or derivative thereof can be generated from nucleic acid from a suitable source. If a clone containing a nucleic acid encoding a particular antibody is not available, but the sequence of the antibody molecule is known, a nucleic acid encoding the antibody can be chemically synthesized or obtained from a suitable source (e.g., an antibody cDNA library, or a cDNA library generated from, or nucleic acid, preferably poly A+ RNA, isolated from, any tissue or cells expressing the antibody or such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

Once the nucleotide sequence and corresponding amino acid sequence of an anti-Pseudomonas Psl and PcrV bispecific binding molecule, e.g., a bispecific antibody or fragment, variant or derivative thereof is determined, its nucleotide sequence can be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an anti-Pseudomonas Psl and PcrV bispecific binding molecule, e.g., a bispecific antibody or fragment, variant or derivative thereof can be composed of any polyribonucleotide or polydeoxribonucleotide, which can be unmodified RNA or DNA or modified RNA or DNA. For example, a polynucleotide encoding an anti-Pseudomonas Psl and PcrV bispecific binding molecule, e.g., a bispecific antibody or fragment, variant or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, a polynucleotide encoding an anti-Pseudomonas Psl and PcrV bispecific binding molecule, e.g., a bispecific antibody or fragment, variant or derivative thereof can be composed of triple-stranded regions comprising RNA or DNA or both RNA and DNA. A polynucleotide encoding an anti-Pseudomonas Psl and PcrV bispecific binding molecule, e.g., a bispecific antibody or fragment, variant or derivative thereof can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations can be introduced by standard techniques, such as site-directed mutagenesis and PCR-mediated mutagenesis. Conservative amino acid substitutions are made at one or more non-essential amino acid residues.

### VI. Expression of Antibody Polypeptides

As is well known, RNA can be isolated from the original hybridoma cells or from other transformed cells by standard techniques, such as guanidinium isothiocyanate extraction and precipitation followed by centrifugation or chromatography. Where desirable, mRNA can be isolated from total RNA by standard techniques such as chromatography on oligo dT cellulose. Suitable techniques are familiar in the art.

In one embodiment, cDNAs that encode the light and the heavy chains of the anti-Pseudomonas Psl and PcrV bispecific binding molecule, e.g., a bispecific antibody or fragment, variant or derivative thereof can be made, either simultaneously or separately, using reverse transcriptase and DNA polymerase in accordance with well-known methods. PCR can be initiated by consensus constant region primers or by more specific primers based on the published heavy and light chain DNA and amino acid sequences. As discussed above, PCR also can be used to isolate DNA clones encoding the antibody light and heavy chains. In this case the libraries can be screened by consensus primers or larger homologous probes, such as mouse constant region probes.

DNA, typically plasmid DNA, can be isolated from the cells using techniques known in the art, restriction mapped and sequenced in accordance with standard, well known techniques set forth in detail, e.g., in the foregoing references relating to recombinant DNA techniques. Of course, the DNA can be synthetic according to the present disclosure at any point during the isolation process or subsequent analysis.

Following manipulation of the isolated genetic material to provide an anti-Pseudomonas Psl and PcrV bispecific binding molecule, e.g., antibody or fragment, variant or derivative thereof of the disclosure, the polynucleotides encoding anti-Pseudomonas Psl and PcrV bispecific binding domains are typically inserted in an expression vector for introduction into host cells that can be used to produce the desired quantity of anti-Pseudomonas Psl and PcrV bispecific binding molecules.

Recombinant expression of a bispecific antibody, or fragment, derivative or analog thereof, e.g., a heavy or light chain of a bispecific antibody that binds to the target molecules described herein, Psl and PcrV, requires construction of an expression vector, or two or more expression vectors, containing a polynucleotide that encodes the antibody. Once a polynucleotide encoding a bispecific antibody molecule or a heavy or light chain of a bispecific antibody, or portion thereof (containing the heavy or light chain variable domain), of the disclosure has been obtained, the vector (or vectors) for the production of the bispecific antibody molecule can be produced by recombinant DNA technology using techniques well known in the art. Thus, methods for preparing a protein by expressing a polynucleotide containing a bispecific antibody-encoding nucleotide sequence are described herein. Methods well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The disclosure, thus, provides replicable vectors comprising a nucleotide sequence encoding a bispecific antibody molecule of the disclosure, or a heavy or light chain thereof, or a heavy or light chain variable domain, operably linked to a promoter. Such vectors can include the nucleotide sequence encoding the constant region of the bispecific antibody molecule (see, e.g., PCT Publication WO 86/05807; PCT Publication WO 89/01036; and U.S. Pat. No. 5,122,464) and the variable domain of the bispecific antibody can be cloned into such a vector for expression of the entire heavy or light chain.

The term “vector” or “expression vector” is used herein to mean vectors used in accordance with the present disclosure as a vehicle for introducing into and expressing a desired gene in a host cell. As known to those skilled in the art, such vectors can easily be selected from the group consisting of plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

For the purposes of this disclosure, numerous expression vector systems can be employed. For example, one class of vector utilizes DNA elements that are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells that have integrated the DNA into their chromosomes can be selected by introducing one or more markers that allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements can also be needed for optimal synthesis of mRNA. These elements can include signal sequences, splice signals, as well as transcriptional promoters, enhancers, and termination signals.

In some embodiments the cloned variable region genes are inserted into an expression vector along with the heavy and light chain constant region genes (e.g., human), e.g., in the Bs1, Bs2, Bs3, or Bs4 formats as discussed above. Of course, any expression vector that is capable of eliciting expression in eukaryotic cells can be used in the present disclosure. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6N5-His, pVAX1, and pZeoSV2 (available from Invitrogen, San Diego, Calif.), and plasmid pCI (available from Promega, Madison, Wis.). In general, screening large numbers of transformed cells for those that express suitably high levels if immunoglobulin heavy and light chains is routine experimentation that can be carried out, for example, by robotic systems.

More generally, once the vector or DNA sequence encoding a monomeric subunit of an anti-Pseudomonas Psl and PcrV bispecific binding molecule, e.g., a bispecific antibody or fragment, variant or derivative thereof of the disclosure has been prepared, the expression vector can be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector can be transferred to a host cell by conventional techniques and the transfected cells can then be cultured by conventional techniques to produce an antibody for use in the methods described herein. Thus, the disclosure includes host cells containing a polynucleotide encoding an anti-Pseudomonas Psl and PcrV bispecific binding molecule, e.g., a bispecific antibody or fragment, variant or derivative thereof, or a heavy or light chain thereof, operably linked to a heterologous promoter. In some embodiments for the expression of double-chained antibodies, vectors encoding both the heavy and light chains can be co-expressed in the host cell for expression of the entire immunoglobulin molecule, as detailed below.

Certain embodiments include an isolated polynucleotide comprising a nucleic acid that encodes the above-described heavy and light chains, wherein a bispecific binding molecule or antigen-binding fragment thereof expressed by the polynucleotide specifically binds Pseudomonas Psl and PcrV.

Some embodiments include vectors comprising the above-described polynucleotides. In further embodiments, the polynucleotides are operably associated with a promoter. In additional embodiments, the disclosure provides host cells comprising such vectors. In further embodiments, the disclosure provides vectors where the polynucleotide is operably associated with a promoter, wherein vectors can express a bispecific binding molecule that specifically binds Pseudomonas Psl and PcrV in a suitable host cell.

Also provided is a method of producing a bispecific binding molecule or fragment thereof that specifically binds Pseudomonas Psl and PcrV, comprising culturing a host cell containing a vector comprising the above-described polynucleotides, and recovering said antibody, or fragment thereof. In further embodiments, the disclosure provides an isolated binding molecule or fragment thereof produced by the above-described method.

As used herein, “host cells” refers to cells that harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” can mean either from spun down whole cells, or from the cell culture containing both the medium and the suspended cells.

A variety of host-expression vector systems can be utilized to express antibody molecules for use in the methods described herein. Such host-expression systems represent vehicles by which the coding sequences of interest can be produced and subsequently purified, but also represent cells that can, when transformed or transfected with the appropriate nucleotide coding sequences, express a bispecific antibody molecule of the disclosure in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

The host cell line used for protein expression is often of mammalian origin; those skilled in the art are credited with ability to determine particular host cell lines that are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

In addition, a host cell strain can be chosen that modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used.

For long-term, high-yield production of recombinant proteins, stable expression can be used. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines that stably express the antibody molecule.

A number of selection systems can be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology that can be used are described in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds), Current Protocols in Human Genetics, John Wiley & Sons, N Y (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Constructs encoding anti-Pseudomonas Psi and PcrV bispecific binding molecules, e.g., bispecific antibodies or fragments, variants or derivatives thereof, as disclosed herein can also be expressed non-mammalian cells such as bacteria or yeast or plant cells. Bacteria that readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors can be advantageously selected depending upon the use intended for the antibody molecule being expressed. For example, when a large quantity of such a protein is to be produced, for the generation of pharmaceutical compositions of an antibody molecule, vectors that direct the expression of high levels of fusion protein products that are readily purified can be desirable. Such vectors include, but are not limited, to the E. coli expression vector pUR278 (Ruther et al., EMBO J. 2:1791 (1983)), in which the antibody coding sequence can be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors can also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes can also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene that provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

Once the anti-Pseudomonas Psl and PcrV bispecific binding molecule, e.g., bispecific antibody or fragment, variant or derivative thereof, as disclosed herein has been recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Another method for increasing the affinity of antibodies of the disclosure is disclosed in US 2002 0123057 A1.

### VII. Pharmaceutical Compositions Comprising Anti-Pseudomonas Psl and PcrV Bispecific Binding Molecules

The pharmaceutical compositions used in this disclosure comprise pharmaceutically acceptable carriers well known to those of ordinary skill in the art. Preparations for parenteral administration include sterile aqueous or non-aqueous solutions, suspensions, and emulsions. Certain pharmaceutical compositions as disclosed herein can be orally administered in an acceptable dosage form including, e.g., capsules, tablets, aqueous suspensions or solutions. Certain pharmaceutical compositions also can be administered by nasal aerosol or inhalation. Preservatives and other additives can also be present such as for example, antimicrobials, antioxidants, chelating agents, and inert gases and the like. Suitable formulations for use in the therapeutic methods disclosed herein are described in Remington's Pharmaceutical Sciences, Mack Publishing Co., 16th ed. (1980).

The amount of an anti-Pseudomonas Psl and PcrV bispecific binding molecule, e.g., a bispecific antibody or fragment, variant or derivative thereof, that can be combined with the carrier materials to produce a single dosage form will vary depending upon the host treated and the particular mode of administration. Dosage regimens also can be adjusted to provide the optimum desired response (e.g., a therapeutic or prophylactic response). The compositions can also comprise anti-Pseudomonas Psl and PcrV bispecific binding molecules, e.g., bispecific antibodies or fragments, variants or derivatives thereof dispersed in a biocompatible carrier material that functions as a suitable delivery or support system for the compounds.

### VIII. Treatment Methods Using Therapeutic Binding Molecules

Methods of preparing and administering an anti-Pseudomonas Psl and PcrV bispecific binding molecule, e.g., a bispecific antibody or fragment, variant or derivative thereof, as disclosed herein to a subject in need thereof are well known to or are readily determined by those skilled in the art. The route of administration of an anti-Pseudomonas Psl and PcrV bispecific binding molecule, e.g., a bispecific antibody or fragment, variant or derivative thereof, can be, for example, oral, parenteral, by inhalation or topical. The term parenteral as used herein includes, e.g., intravenous, intraarterial, intraperitoneal, intramuscular, or subcutaneous administration. A suitable form for administration would be a solution for injection, in particular for intravenous or intraarterial injection or drip. However, in other methods compatible with the teachings herein, an anti-Pseudomonas Psl and PcrV bispecific binding molecule, e.g., a bispecific antibody or fragment, variant or derivative thereof, as disclosed herein can be delivered directly to the site of the adverse cellular population e.g., a Pseudomonas infection, thereby increasing the exposure of the diseased tissue to the therapeutic agent. For example, an anti-Pseudomonas Psl and PcrV bispecific binding molecule can be directly administered to ocular tissue, burn injury, or lung tissue.

Anti-Pseudomonas Psl and PcrV bispecific binding molecules, e.g., bispecific antibodies or fragments, variants or derivatives thereof, as disclosed herein can be administered in a pharmaceutically effective amount for the in vivo treatment of Pseudomonas infection. In this regard, it will be appreciated that the disclosed binding molecules will be formulated so as to facilitate administration and promote stability of the active agent. For the purposes of the instant disclosure, a pharmaceutically effective amount shall be held to mean an amount sufficient to achieve effective binding to a target and to achieve a benefit, e.g., treat, ameliorate, lessen, clear, or prevent Pseudomonas infection.

Some embodiments are directed to a method of preventing or treating a Pseudomonas infection in a subject in need thereof, comprising administering to the subject an effective amount of the bispecific binding molecule or fragment thereof, the bispecific antibody or fragment thereof, the composition, the polynucleotide(s), the vector(s), or the host cell(s) described herein. Some embodiments are directed to use of a bispecific binding molecule or fragment thereof, a bispecific antibody or fragment thereof, a composition, polynucleotide(s), vector(s), or host cell(s) described herein in the preparation of a medicament for the treatment of Pseudomonas infection. Some embodiments are directed to a bispecific antibody or fragment thereof, a composition, polynucleotide(s), vector(s), or host cell(s) described herein for the treatment of a Pseudomonas infection. In further embodiments, the Pseudomonas infection is a P. aeruginosa infection. In some embodiments, the subject is a human. In certain embodiments, the infection is an ocular infection, a lung infection, a burn infection, a wound infection, a skin infection, a blood infection, a bone infection, or a combination of two or more such infections. In further embodiments, the subject suffers from acute pneumonia, burn injury, corneal infection, cystic fibrosis, or a combination thereof.

Certain embodiments are directed to a method of blocking or preventing attachment of P. aeruginosa to epithelial cells comprising contacting a mixture of epithelial cells and P. aeruginosa with the bispecific binding molecule or fragment thereof, the bispecific antibody or fragment thereof, the composition, the polynucleotide(s), the vector(s), or the host cell(s) described herein. Some embodiments are directed to use of a bispecific binding molecule or fragment thereof, a bispecific antibody or fragment thereof, a composition, polynucleotide(s), vector(s), or host cell(s) described herein in the preparation of a medicament for the blocking or preventing attachment of P. aeruginosa to epithelial cells. Some embodiments are directed to a bispecific antibody or fragment thereof, a composition, polynucleotide(s), vector(s), or host cell(s) described herein for the blocking or preventing attachment of P. aeruginosa to epithelial cells.

Also disclosed is a method of enhancing OPK of P. aeruginosa comprising contacting a mixture of phagocytic cells and P. aeruginosa with the bispecific binding molecule or fragment thereof, the bispecific antibody or fragment thereof, the composition, the polynucleotide(s), the vector(s), or the host cell(s) described herein. Some embodiments are directed to use of a bispecific binding molecule or fragment thereof, a bispecific antibody or fragment thereof, a composition, polynucleotide(s), vector(s), or host cell(s) described herein in the preparation of a medicament for enhancing OPK of P. aeruginosa. Some embodiments are directed to a bispecific antibody or fragment thereof, a composition, polynucleotide(s), vector(s), or host cell(s) described herein for enhancing OPK of P. aeruginosa. In further embodiments, the phagocytic cells are differentiated HL-60 cells or human polymorphonuclear leukocytes (PMNs).

In keeping with the scope of the disclosure, anti-Pseudomonas Psl and PcrV bispecific binding molecules, e.g., bispecific antibodies or fragments, variants or derivatives thereof, can be administered to a human or other animal in accordance with the aforementioned methods of treatment in an amount sufficient to produce a therapeutic effect. The anti-Pseudomonas Psl and PcrV bispecific binding molecules, e.g., bispecific antibodies or fragments, variants or derivatives thereof, disclosed herein can be administered to such human or other animal in a conventional dosage form prepared by combining the antibody of the disclosure with a conventional pharmaceutically acceptable carrier or diluent according to known techniques.

Effective doses of the compositions of the present disclosure, for treatment of Pseudomonas infection vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

Anti-Pseudomonas Psl and PcrV bispecific binding molecules, e.g., bispecific antibodies or fragments, variants or derivatives thereof can be administered multiple occasions at various frequencies depending on various factors known to those of skill in the art. Alternatively, anti-Pseudomonas Psl and PcrV bispecific binding molecules, e.g., bispecific antibodies or fragments, variants or derivatives thereof can be administered as a sustained release formulation, in which case less frequent administration is required. Dosage and frequency vary depending on the half-life of the antibody in the patient.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

### IX. Synergy

Chou and Talalay (Adv. Enzyme Regul., 22:27-55 (1984)) developed a mathematical method to describe the experimental findings of combined drug effects in a qualitative and quantitative manner. For mutually exclusive drugs, they showed that the generalized isobol equation applies for any degree of effect (see page 52 in Chou and Talalay). An isobol or isobologram is the graphic representation of all dose combinations of two drugs that have the same degree of effect. In isobolograms, a straight line indicates additive effects, a concave curve (curve below the straight line) represents synergistic effects, and a convex curve (curve above the straight line) represents antagonistic effects. These curves also show that a combination of two mutually exclusive drugs will show the same type of effect over the whole concentration range, either the combination is additive, synergistic, or antagonistic. Most drug combinations show an additive effect. In some instances however, the combinations show less or more than an additive effect. These combinations are called antagonistic or synergistic, respectively. A combination manifests therapeutic synergy if it is therapeutically superior to one or other of the constituents used at its optimum dose. See, T. H. Corbett et al., Cancer Treatment Reports, 66, 1187 (1982). Tallarida R J (J Pharmacol Exp Ther. 2001 September; 298 (3):865-72) also notes “Two drugs that produce overtly similar effects will sometimes produce exaggerated or diminished effects when used concurrently. A quantitative assessment is necessary to distinguish these cases from simply additive action.”

A synergistic effect can be measured using the combination index (CI) method of Chou and Talalay (see Chang et al., Cancer Res. 45: 2434-2439, (1985)), which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. In another embodiment, a synergistic effect is determined by using the fractional inhibitory concentration (FIC). This fractional value is determined by expressing the IC50 of a drug acting in combination, as a function of the IC50 of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

In some embodiments, a synergistic effect is obtained in Pseudomonas treatment wherein one or more of the binding agents are administered in a “low dose” (i.e., using a dose or doses that would be considered non-therapeutic if administered alone), wherein the administration of the low dose binding agent in combination with other binding agents (administered at either a low or therapeutic dose) results in a synergistic effect that exceeds the additive effects that would otherwise result from individual administration of the binding agent alone. In some embodiments, the synergistic effect is achieved via administration of one or more of the binding agents administered in a “low dose” wherein the low dose is provided to reduce or avoid toxicity or other undesirable side effects.

### X. Immunoassays

Anti-Pseudomonas Psl and PcrV bispecific binding molecules, e.g., bispecific antibodies or fragments, variants or derivatives thereof can be assayed for immunospecific binding by any method known in the art. The immunoassays that can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities. Antibody affinity can be measured by a number of methods, including OCTET®, BIACORE®, ELISA, and FACS.

The OCTET® system uses biosensors in a 96-well plate format to report kinetic analysis. Protein binding and dissociation events can be monitored by measuring the binding of one protein in solution to a second protein immobilized on the FortéBio biosensor. In the case of measuring binding of anti-Psl or PcrV antibodies to Psl or PcrV, the Psl or PcrV is immobilized onto OCTET® tips followed by analysis of binding of the antibody, which is in solution. Association and disassociation of antibody to immobilized Psl or PcrV is then detected by the instrument sensor. The data is then collected and exported to GraphPad Prism for affinity curve fitting.

Surface plasmon resonance (SPR) as performed on BIACORE® offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE® code No. BR-1001-86; BIAtechnology Handbook, version AB (reprinted 1998), BIACORE® code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIACORE® measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIACORE® investigations. The range of affinities measurable by this technique is very broad ranging from mM to pM.

Epitope specificity is an important characteristic of a monoclonal antibody. Epitope mapping with BIACORE®, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIACORE® are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides that interfere with binding of a given MAb are assumed to be structurally related to the epitope defined by that MAb.

### XI. Kits

In yet other embodiments, the present invention provides kits that can be used to perform the methods described herein. In certain embodiments, a kit comprises a binding molecule disclosed herein in one or more containers. One skilled in the art will readily recognize that the disclosed binding domains, polypeptides and antibodies of the present invention can be readily incorporated into one of the established kit formats that are well known in the art.

The practice of the disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlag (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

### EXAMPLES

### Example 1: Construction of WapR-004/V2L2 Bispecific Antibodies and Related Antibodies

Since the combination of WapR-004+V2L2 provided protection against Pseudomonas challenge (see, e.g., PCT Application No. PCT/US2013/068609), bispecific constructs were generated. Production of the constructs begun with template bispecific antibodies binding to TNF-alpha (IgG) and ANG-2 (ScFv).

The building blocks for these bispecific antibodies were disclosed in PCT Application No. PCT/US2013/068609. These include anti-Psl antibodies WapR-004 (W4), W4-RAD, W4-RAD-2C, and Psl0096, both in IgG format and in ScFv format, and anti-PcrV antibodies V2L2, and V2L2-MD. The VH and VL amino acid sequences of these antibodies are presented in Table 2.

TABLE 2
Amino acid sequences of building block antibodies

| Antibody Name | VH | VL |
|---|---|---|
| WapR-004 | EVQLLESGPGLVKPSET | EIVLTQSPSSLSTSVGDRVTITCRASQ |
| (W4) | LSLTCNVAGGSISPYYW | SIRSHLNWYQQKPGKAPKLLIYGASN |
| | TWIRQPPGKGLELIGYI | LQSGVPSRFSGSGSGTDFTLTISSLQP |
| | HSSGYTDYNPSLKSRVT | EDFATYYCQQSYSFPLTFGGGTKLEI |
| | ISGDTSKKQFSLHVSSV | K (SEQ ID NO: 15) |
| | TAADTAVYFCARGDW | |
| | DLLHALDIWGQGTLVT | |
| | VSS (SEQ ID NO: 14) | |
| WapR- | EVQLLESGPGLVKPSET | EIVLTQSPSSLSTSVGDRVTITCRASQ |
| 004RAD | LSLTCNVAGGSISPYYW | SIRSHLNWYQQKPGKAPKLLIYGASN |
| (W4-RAD) | TWIRQPPGKGLELIGYI | LQSGVPSRFSGSGSGTDFTLTISSLQP |
| | HSSGYTDYNPSLKSRVT | EDFATYYCQQSYSFPLTFGGGTKLEI |
| | ISGDTSKKQFSLHVSSV | K (SEQ ID NO: 15) |
| | TAADTAVYFCARADW | |
| | DLLHALDIWGQGTLVT | |
| | VSS (SEQ ID NO: 16) | |
| WapR- | EVQLLESGPGLVKPSET | EIVLTQSPSSLSTSVGDRVTITCRASQ |
| 004RAD-2C | LSLTCNVAGGSISPYYW | SIRSHLNWYQQKPGKAPKLLIYGASN |
| (W4-RAD- | TWIRQPPGKCLELIGYI | LQSGVPSRFSGSGSGTDFTLTISSLQP |
| 2C) | HSSGYTDYNPSLKSRVT | EDFATYYCQQSYSFPLTFGCGTKLEI |
| | ISGDTSKKQFSLHVSSV | K (SEQ ID NO: 18) |
| | TAADTAVYFCARADW | |
| | DLLHALDIWGQGTLVT | |
| | VSS (SEQ ID NO: 17) | |
| Psl0096 | EVQLLESGPGLVKPSET | EIVLTQSPSSLSTSVGDRVTITCRASQ |
| | LSLTCNVAGGSISPYYW | SIRSHLNWYQQKPGKAPKLLIYGASN |
| | TWIRQPPGKCLELIGYI | LQSGVPSRFSGSGSGTDFTLTISSLQP |
| | HSSGYTDYNPSLKSRVT | EDFATYYCQQSYSFPLTFGCGTKLEI |
| | ISGDTSKKQFSLHVSSV | K (SEQ ID NO: 18) |
| | TAADTAVYFCARADW | |
| | DLLHALDIWGQGTLVT | |
| | VSS (SEQ ID NO: 17) | |
| V2L2 | EMQLLESGGGLVQPGG | AIQMTQSPSSLSASVGDRVTITCRAS |
| | SLRLSCAASGFTFSSYA | QGIRNDLGWYQQKPGKAPKLVIYSA |
| | MNWVRQAPGEGLEWV | STLQSGVPSRFSGSGSGTDFTLSISSL |
| | SAITISGITAYYTDSVKG | QPDDFATYYCLQDYNYPWTFGQGTK |
| | RFTISRDNSKNTLYLQM | VEIK (SEQ ID NO: 20) |
| | NSLRAGDTAVYYCAKE | |
| | EFLPGTHYYYGMDVW | |
| | GQGTTVTVSS (SEQ ID | |
| | NO: 19) | |
| V2L2-MD | EMQLLESGGGLVQPGG | AIQMTQSPSSLSASVGDRVTITCRAS |
| | SLRLSCAASGFTFSSYA | QGIRNDLGWYQQKPGKAPKLLIYSA |
| | MNWVRQAPGEGLEWV | STLQSGVPSRFSGSGSGTDFTLTISSL |
| | SAITISGITAYYTDSVKG | QPEDFATYYCLQDYNYPWTFGQGTK |
| | RFTISRDNSKNTLYLQM | VEIK (SEQ ID NO: 21) |
| | NSLRAGDTAVYYCAKE | |
| | EFLPGTHYYYGMDVW | |
| | GQGTTVTVSS (SEQ ID | |
| | NO: 19) | |

FIG. 1A shows TNFα bispecific model constructs. For Bs1-TNFα/W4, the W4 scFv was fused to the amino-terminus of TNFα VL through a (G4S)₂ linker (SEQ ID NO: 1). For Bs2-TNFα/W4, the W4 scFv was fused to the amino-terminus of TNFα VH through a (G4S)₂ linker (SEQ ID NO: 1). For Bs3-TNFα/W4, the W4 scFv was fused to the carboxy-terminus of CH3 through a (G4S)₂ linker (SEQ ID NO: 1).

Since the combination of WapR-004+V2L2 provide protection against Pseudomonas challenge, bispecific constructs were generated comprising a WapR-004 scFv (W4-RAD) and V2L2 IgG (FIG. 1B).

### Bs1-TNFαW4 Construction:

W4 scFv for Bs1 vector was amplified by PCR. The following primers were used to amplify W4-VH for Bs1: W4-VH forward primer: TGGCTCCCCGGGGCgcgcTGTGAGGTGCAGCTGTTGGAGTCGG (SEQ ID NO: 22), W4-VH reverse primer: CTCCGCCACTCGAGACGGTGACCAGGGTCC (SEQ ID NO: 23). The template for VH PCR amplification was pEU-W4-HC, a vector containing W4 heavy chain. For amplification of the light chain the following primers were used: W4-VL forward primer 1: ACCGTCTCGA GTGGCGGAGG GGGCTCTGGG GGAGGGGGCA GCGGCGGCGG AGGATCTG (SEQ ID NO: 24) (W4-VL forward primer 2: AGCGGCGGCG GAGGATCTGG GGGAGGGGGC AGCGAAATTG TGTTGACACA GTCTCCATC (SEQ ID NO: 25); and W4-VL reverse primer: GCCCCCTCCG CCGGATCCCC CTCCGCCTTT GATCTCCAGC TTGGTCCCTCC (SEQ ID NO: 26). The template for VL PCR amplification was pEU-W4-LC, the vector containing W4 light chain. The two forward primers and the reverse primer were used in a single reaction to accomplish a 5′ extension that would have been excessively long for a single primer. Primer ratios were 5:1:1 (fwd1:fwd2:rev).

After PCR amplification, VH and VL bands were gel purified. Bs1-TNFα/Ang2 (constructed previously) was digested with BssHII/BamHI, the vector band was gel purified, and assembled with W4-VH-linker-W4-VL-linker for Bs1 vector by using the IN-FUSION® system (Clontech), then transformed into STELLAR™ competent cells (Clontech), colonies were sequenced for correct W4 scFv insert.

### Bs2-TNFα/W4 Construction:

W4 scFv for Bs2 vector was amplified by PCR. The following primers were used to amplify W4-VH and W4-VL for Bs2: W4-VH forward primer: TTCTCTCCAC AGGTGTaCAc tccGAGGTGC AGCTGTTGGA GTCGG (SEQ ID NO: 27); and W4-VH reverse primer: CTCCGCCACT CGAGACGGTG ACCAGGGTCC (SEQ ID NO: 28). The template for VH PCR amplification was pEU-W4-HC, the vector containing W4 heavy chain. For amplification of the light chain the following primers were used: W4-VL forward primer 1: W4-VL forward primer 1: ACCGTCTCGA GTGGCGGAGG GGGCTCTGGG GGAGGGGGCA GCGGCGGCGG AGGATCTG (SEQ ID NO: 24); W4-VL forward primer 2: AGCGGCGGCG GAGGATCTGG GGGAGGGGGC AGCGAAATTG TGTTGACACA GTCTCCATC (SEQ ID NO: 25); and W4-VL reverse primer: GCCCCCTCCG CCGGATCCCC CTCCGCCTTT GATCTCCAGC TTGGTCCCTCC (SEQ ID NO: 26). The template for VL PCR amplification was pEU-W4-LC, the vector containing W4 light chain. The two forward primers and the reverse primer were used in a single reaction to accomplish a 5′ extension that would have been excessively long for a single primer. Primer ratios were 5:1:1 (fwd1:fwd2:rev). After PCR amplification, VH and VL bands were gel purified. Bs2-TNFa/Ang2 (constructed previously) was digested with BsrGI/BamHI, the vector band was gel purified, and assembled with W4-VH-linker-W4-VL-linker for the Bs2 vector by using the IN-FUSION®® system, then transformed STELLAR™™ competent cells, colonies were sequenced for correct W4 scFv insert. Bs3-TNFa/W4 construction:

W4 scFv for Bs3 vector was amplified by PCR. The following primers were used to amplify W4-VH and W4-VL for Bs3: W4-VH forward primer: GTAAAGGCGG AGGGGGATCC GGCGGAGGGG GCTCTGAGGT GCAGCTGTTG GAGTCGG (SEQ ID NO: 29); and W4-VH reverse primer: CTCCGCCACT CGAGACGGTG ACCAGGGTCC (SEQ ID NO: 28). The template for VH PCR amplification was pEU-W4-HC, the vector containing W4 heavy chain. For amplification of the light chain the following primers were used: W4-VL forward primer 1: W4-VL forward primer 1: W4-VL forward primer 1: ACCGTCTCGA GTGGCGGAGG GGGCTCTGGG GGAGGGGGCA GCGGCGGCGG AGGATCTG (SEQ ID NO: 24); W4-VL forward primer 2: AGCGGCGGCG GAGGATCTGG GGGAGGGGGC AGCGAAATTG TGTTGACACA GTCTCCATC (SEQ ID NO: 25); and W4-VL reverse primer: GATCAATGAA TTCGCGGCCG CTCATTTGAT CTCCAGCTTG GTCCCTCCG (SEQ ID NO: 30). The template for VL PCR amplification was pEU-W4-LC, the vector containing W4 light chain. The 2 forward primers and the reverse primers were used in a single reaction to accomplish a 5′ extension that would have been excessively long for a single primer. Primer ratios were 5:1:1 (fwd1:fwd2:rev). After PCR amplification, VH and VL bands were gel purified. Bs3-TNFa/Ang2 (constructed previously) was digested with BamHI/NotI, vector band was gel purified, and assembled with linker-W4-VH-linker-W4-VL for Bs3 vector by using the IN-FUSION® system, then transformed STELLAR™ competent cells, colonies were sequenced for correct W4 scFv insert.

### Bs2-V2L2/W4 Construction:

V2L2-VL and VH for Bs2 vector were amplified by PCR, following primers were used to amplify. V2L2-VL forward primer: TGGCTCCCCG GGGCgcgcTG TGCCATCCAG ATGACCCAGT CTCC (SEQ ID NO: 31); V2L2-VL reverse primer: TGGTGCAGCC ACCGTACGTT TGATTTCAAC CTTGGTCCCT TG (SEQ ID NO: 32); V2L2-VH forward primer: AAGGCGGAGG GGGATCCGGC GGAGGGGGCT CTGAGATGCA GCTGTTGGAG TCTGG (SEQ ID NO: 33); and V2L2-VH reverse primer: GATGGGCCCT TGGTcGAcGC TGAGGAGACG GTGACCGTGG TCC (SEQ ID NO: 34). After PCR amplification, VH and VL bands were gel purified. Bs2-TNFa/W4 was digested with BssHII/BsiWI, vector band was gel purified, and assembled with V2L2-VL by using the IN-FUSION® system, then transformed into STELLAR™ competent cells, colonies were sequenced for correct V2L2-VL insert. Then this vector Bs2-V2L2-VL/W4 was digested with BsrGI/SalI, the vector band was gel purified, ligated with W4-scFv and V2L2-VH by using the IN-FUSION® system, transformed STELLAR™ competent cells, colonies were sequenced for the correct W4 scFv-V2L2 VH insert.

### Bs3-V2L2/W4 Construction:

The pOE-V2L2 IgG vector was digested with BssHII/SalI, and the insert band, containing V2L2 LC and V2L2-VH, was gel purified, Bs3-TNFa/W4 was also digested with BssHII/SalI, and vector band was purified and ligated with V2L2 insert, then transformed into stable 3 competent cells. Colonies were sequenced for correct V2L2-VL and VH insert.

### Construction of Bs2-V2L2/W4-RAD-2C, Bs3-V2L2/W4-RAD-2C and Bs4-V2L2/W4-RAD-2C

To generate Bs2-V2L2-2C, the W4-RAD-2C scFv was fused to N-terminus1 of V2L2 VH through a (G4S)₂ linker (SEQ ID NO: 1). To generate Bs3-V2L2-2C, the W4-RAD-2C scFv was fused to C-terminal of CH3 through (G4S)₂ linker (SEQ ID NO: 1). To generate Bs4-V2L2-2C, the W4-RAD-2C scFv was inserted in the hinge region, linked by (G4S)₂ linkers (SEQ ID NO: 1) on the N-terminus and C-terminus1 of the scFv. To generate Bs2-W4-RAD-2C, a V2L2 scFv was fused to the amino-terminus of W4-RAD VH through a (G4S)₂ linker (SEQ ID NO: 1).

To generate the W4-RAD scFv for the Bs3 construct, the W4-RAD-2C scFv containing 3 mutations (cysteine mutations in VH-44 and VL-100 to form the di-sulfide bond between VH and VL and G to A mutation in VH-95 to remove RGD motif, called RAD) was amplified by PCR. Three pieces of the W4-RAD scFv were amplified, the template was W4 scFv, then overlapped to W4-RAD scFv. Primers used to amplify the 1st piece of the W4-RAD scFv were: W4 VH forward primer: (includes 5 bp of CH3, (G4S)2 linker (SEQ ID NO: 1) and 22 bp of VH N-terminal sequence) GTAAAGGCGG AGGGGGATCC GGCGGAGGGG GCTCTGAGGT GCAGCTGTTG GAGTCGG (SEQ ID NO: 29), and the W4 VH cysteine mutation reverse primer: CAACTCCAGG CACTTCCCTGG (SEQ ID NO: 35); primers for the 2nd piece of W4-RAD scFv were: W4 VH cysteine mutation forward primer: CCAGGGAAGT GCCTGGAGTTG (SEQ ID NO: 36) and W4 VH RAD mutation reverse primer: GTCCCAATCG GCTCTCGCACAG (SEQ ID NO: 37); primers for 3rd piece of W4-RAD scFv were: W4 VH RAD mutation forward primer: CTGTGCGAGA GCCGATTGGGAC (SEQ ID NO: 38) and W4 VL reverse primer: (includes part of vector sequence and 32 bp of VL C-terminal sequence including cysteine mutation at VL-100) CAATGAATTC GCGGCCGCTC ATTTGATCTC CAGCTTGGTC CCACAGCCGA AAG (SEQ ID NO: 39). The overlapping fragments were then fused together to form the W4-RAD-2C scFv.
W4-RAD scFv sequence in Bs3 vector: underlined sequences are G4S linker (SEQ ID NO: 40):

(SEQ ID NO: 41)
GGGGSGGGGSEVQLLESGPGLVKPSETLSLTCNVAGGSISPYYWTWIRQP
PGKCLELIGYIHSSGYTDYNPSLKSRVTISGDTSKKQFSLHVSSVTAADT
AVYFCARADWDLLHALDIWGQGTLVTVSSGGGGSGGGGSGGGGSGGGGSE
IVLTQSPSSLSTSVGDRVTITCRASQSIRSHLNWYQQKPGKAPKLLIYGA
SNLQSGVPSRFSGSGSGTDFTLTISSLQPEDFATYYCQQSYSFPLTFGCG
TKLEIK

After the W4-RAD-2C scFv fragment was amplified, it was then gel purified and ligated into the Bs3 vector: Bs3-V2L2/W4 was digested with BamHI/NotI, the vector band containing V2L2 IgG portion was gel purified, the Bs3-V2L2 vector and the W4-RAD-2C scFv were assembled by using the IN-FUSION® system, followed by transformation in STELLAR™ competent cells. Colonies were sequenced to confirm the correct W4-RAD-2C scFv insert and V2L2 VH and VL, this vector was called Bs3-V2L2-2C.

A similar approach was used to generate Bs2-V2L2-2C. W4-RAD scFv-V2L2 VH sequences in the Bs2 vector: underlined sequences are the G4S linker (SEQ ID NO: 40):

(SEQ ID NO: 42)
EVQLLESGPGLVKPSETLSLTCNVAGGSISPYYWTWIRQPPGKCLELIGY
IHSSGYTDYNPSLKSRVTISGDTSKKQFSLHVSSVTAADTAVYFCARADW
DLLHALDIWGQGTLVTVSSGGGGSGGGGSGGGGSGGGGSEIVLTQSPSSL
STSVGDRVTITCRASQSIRSHLNWYQQKPGKAPKLLIYGASNLQSGVPSR
FSGSGSGTDFTLTISSLQPEDFATYYCQQSYSFPLTFGCGTKLEIKGGGG
SGGGGSEMQLLESGGGLVQPGGSLRLSCAASGFTFSSYAMNWVRQAPGEG
LEWVSAITISGITAYYTDSVKGRFTISRDNSKNTLYLQMNSLRAGDTAVY
YCAKEEFLPGTHYYYGMDVWGQGTTVTVSS

The following primers were used to amplify the W4-RAD-2C scFv. VH forward primer and VL reverse primer: W4-RAD-2C VH forward primer for Bs2 vector which includes some intron, 3′ signal peptide and 22 bp of W4-RAD VH N-terminal sequence TTCTCTCCAC AGGTGTACAC TCCGAGGTGC AGCTGTTGGA GTCGG (SEQ ID NO: 27) and W4-RAD VL reverse primer for Bs2 vector: (includes (G4S)2 linker (SEQ ID NO: 1) and 32 bp of VL C-terminal sequence): CCCCCTCCGC CGGATCCCCC TCCGCCTTTG ATCTCCAGCT TGGTCCCACA GCCGAAAG (SEQ ID NO: 43). The three pieces of W4-RAD-2C scFv (as described above for the W4-RAD-2C scFv PCR amplification) and these two primers were overlapped by PCR to form a W4-RAD-2C scFv for the Bs2 vector. W4-RAD scFv (for Bs2) fragment was then gel purified, and ligated into Bs2 vector.

Bs2-V2L2/W4 vector was digested with BsrGI/BamHI, and the vector band was gel purified. The W4-RAD-2C scFv (for Bs2) was then assembled into the Bs2 vector by the IN-FUSION® system and transformed into STELLAR™ competent cells. The colonies were sequence confirmed for the correct W4-RAD-2C scFv, V2L2 VH and VL. This vector was called Bs2-V2L2-2C.

### Bs4-V2L2-2C Construction

The starting construct was the Bs4 vector backbone with G4S linkers (SEQ ID NO: 40), containing a BamHI site in the upper hinge region after the Fab portion of IgG. The hinge region with linker sequence is shown below:

Hinge Region with Linker Sequence:

CH1 hinge linker

(SEQ ID NO:44)

[symbol]

EPKSCGGGGSGGGGS
-N-terminus of scFv C-terminus of scFv-

(SEQ ID NO: 45)

GGGGSGGGGSDKTHTCPPCP [symbol]
CH2 linker hinge

W4-RAD-2C scFv sequences in a BS4 vector: W4-RAD-2C scFv is in bolded italics with the G4S linkers (SEQ ID NO: 40) underlined in bolded italics; the hinge regions are doubled underlined:

(SEQ ID NO: 46)
KVDKRVEPKSCGGGGSGGGGSEVQLLESGPGLVKPSETLSLTCNVAGGSI
SPYYWTWIRQPPGKCLELIGYIHSSGYTDYNPSLKSRVTISGDTSKKQFS
LHVSSVTAADTAVYFCARADWDLLHALDIWGQGTLVTVSSGGGGSGGGGS
GGGGSGGGGSEIVLTQSPSSLSTSVGDRVTITCRASQSIRSHLNWYQQKP
GKAPKLLIYGASNLQSGVPSRFSGSGSGTDFTLTISSLQPEDFATYYCQQ
SYSFPLTFGCGTKLEIKGGGGSGGGGSDKTHTCPPCPAPELL

The following primers were used to amplify the CH1-hinge-linker and linker-hinge-CH2-CH3: CH1-hinge-linker. The forward primer (includes some V2L2 VH C-terminal sequence and CH1 N-terminal sequences): CACGGTCACC GTCTCCTCAG CGTCGACC (SEQ ID NO: 47), and the CH1-hinge-linker reverse primer (includes some linker, hinge and CH1 C-terminal sequences) CCCTCCGCCA GAGCCCCCTC CGCCACAAGA TTTGGGCTCA ACTCTCTTG (SEQ ID NO: 48). The linker-hinge-CH2-CH3 forward primer (includes some linker and hinge sequences): GAGGGGGCTC TGGCGGAGGG GGATCCGACA AAACTCACAC ATGCCCACC (SEQ ID NO: 49), the linker-hinge-CH2-CH3 reverse primer (includes part of vector and CH3 C-terminal sequences): TCAATGAAT TCGCGGCCGC TCATTTACC (SEQ ID NO: 50). pOE-V2L2 IgG vector was used as template of PCR amplification. The PCR amplified CH1-hinge-linker and linker-hinge-CH2-CH3 regions were overlapped by PCR to form a CH1-hinge-linker-hinge-CH2-CH3 fragment. This fragment was then gel purified and ligated with the IgG vector which was digested with SalI/NotI by using the IN-FUSION® system and transformed into STELLAR™ competent cells. The colonies were sequence confirmed for the correct CH1-hinge-linker-hinge-CH2-CH3 sequence. This vector is the Bs4-V2L2 empty vector. The W4-RAD-2C scFv was then ligated into the Bs4 vector to get Bs4-V2L2-2C, by gel purifying the W4-RAD scFv (from PCR). The Bs4-V2L2 vector was digested with BamHI and the vector band was gel purified. The W4-RAD-2C scFv was ligated with the Bs4 vector by the IN-FUSION® system and the vector was used to transform STELLAR™ competent cells. Colonies were sequenced for the correct W4-RAD scFv insert.

The W4-RAD-2C scFv for the Bs4 vector was generated using PCR and the following primers: W4-RAD VH forward primer for Bs4 vector (includes some of linker sequences and 24 bp of W4-RAD VH N-terminal sequence): GAGGTGCAGC TGTTGGAGTC GGGC (SEQ ID NO: 51); and W4-RAD VL reverse primer for Bs4 vector (includes some hinge sequence, linker and 21 bp of W4-RAD VL C-terminal sequence): GTGTGAGTTT TGTCGGATCC CCCTCCGCCA GAGCCACCTC CGCCTTTGAT CTCCAGCTTG GTCCC (SEQ ID NO: 52). Bs2-V2L2/W4-RAD was used as template for the PCR amplification, and the W4-RAD-2C scFv band was then gel purified.

The W4-RAD-2C scFv was then ligated into the Bs4 vector to get Bs4-V2L2-2C: the Bs4-V2L2-empty vector was digested with BamHI and the vector band was gel purified, W4-RAD-2C scFv was assembled with the Bs4 vector by the IN-FUSION® system and was transformed into STELLAR™ competent cells. Colonies were sequenced for the correct W4-RAD scFv insert. This vector was called Bs4-V2L2-2C.

### Bs4-GLO Construction:

Bs4-GLO contains the V2L2MD (germ-lined and lead optimized V2L2) as the IgG arms and a Psl0096 scFv (germ-lined and lead optimized W4) inserted in hinge region.

The plasmid pOE-V2L2MD IgG vector was digested with BssHII/SalI. The insert band (contains V2L2MD LC and V2L2MD-VH) was gel purified. Bs4-V2L2/W4-RAD-2C was also digested with BssHII/SalI, the vector band was purified and ligated with the V2L2MD insert, then transformed Stable 3 competent cells (Life Technologies). Colonies were sequenced for the correct V2L2MD-VL and VH inserts. This vector contains the V2L2MD IgG arm and the W4-RAD-2C scFv.

Then this vector was then digested with BamHI, and the vector band was purified. A Psl0096 scFv containing cysteine in VH-44 and VL-100 positions was synthesized by Operon. The following primers were used to amplify the scFv for Bs4 vector. Psl0096 VH forward primer: TCTGGCGGAG GgggatccCA GGTGCAGCTG CAGGAATCTG GC (SEQ ID NO: 53); and Psl0096 VL reverse primer: GTGAGTTTTG TcggatccCC CTCCGCCAGA GCCACCTCCG CCCTTGATTT CCACCTTGGT GCC (SEQ ID NO: 54). After PCR amplification, the Psl0096 scFv was gel purified and ligated into the Bs4-V2L2MD vector using the IN-FUSION® system, then transformed STELLAR™ competent cells. Colonies were sequenced for the correct Psl0096 scFv insert, and this vector was called Bs4-GLO

### Bs2-GLO Construction:

Bs4-GLO was digested with BssHII/BsiWI, and the V2L2MD-VL insert band was gel purified. The Bs2-V2L2/W4-RAD-2C was also digested with BssHII/BsiWI, the vector band was purified, and then ligated with the V2L2MD-VL fragment. The ligation mix was transformed into Stable 3 competent cells, and colonies were sequenced for correct V2L2MD-VL insert.

This vector Bs2-V2L2MD-VL was then digested with BsrGI/SalI, and the vector band was purified. A Psl0096 scFv for Bs2 vector was amplified by PCR amplification, using the following primers: Psl0096 VH (for Bs2) forward primer: TTCTCTCCAC AGGTGTaCAc tccCAGGTGC AGCTGCAGGA ATCTG (SEQ ID NO: 55); and Psl0096 VL (for Bs2) reverse primer: CCTCCGCCGG ATCCCCCTCC GCCCTTGATT TCCACCTTGG TGCCG (SEQ ID NO: 56). V2L2MD-VH was also amplified by PCR, with the following primers being used for amplification: V2L2MD-VH forward primer: GGGGGATCCG GCGGAGGGGG CTCTGAGGTG CAGCTGTTGG AGTCTGG (SEQ ID NO: 57); and V2L2MD-VH reverse primer: GATGGGCCCT TGGTcGAcGC TGAGGAGACG GTGACCGTGG TCC (SEQ ID NO: 34).

After PCR amplification, the Psl0096 scFv (for Bs2) and V2L2MD-VH fragments were gel purified and ligated into the Bs2-V2L2MD-VL vector using the IN-FUSION® system, then were then transformed using STELLAR™ competent cells. Colonies were sequenced for correct Psl0096 scFv-V2L2MD-VH insert, this vector is called Bs2-GLO.

### Bs3-GLO Construction:

Bs4-GLO was digested with BssHII/SalI, and the insert band containing V2L2MD LC and VH-encoding sequences was gel purified. Bs3-V2L2/W4-RAD-2C was also digested with BssHII/SalI. The vector band was purified, then ligated with the V2L2MD fragment. The ligation mix was used to transform Stable 3 competent cells, and colonies were sequenced for the correct V2L2MD insert. This vector, Bs3-V2L2MD, was then digested with BamHI/NotI, and vector band was gel purified. The Psl0096 scFv for Bs3 vector was amplified by PCR amplification. The ollowing primers were used to amplify the scFv for Bs3 vector Psl0096 VH (for Bs3) forward primer: AAAGGCGGAG GGGGATCCGG CGGAGGGGGC TCTCAGGTGC AGCTGCAGGA ATCTG (SEQ ID NO: 58); and Psl0096 VL (for Bs3) reverse primer: TCAATGAATT CGCGGCCGCT CACTTGATTT CCACCTTGGT GCCGC (SEQ ID NO: 59). After PCR amplification, the Psl0096 scFv (for Bs3) fragment was gel purified and assembled with the Bs3-V2L2MD vector using the IN-FUSION® system, and was then transformed into STELLAR™ competent cells. Colonies were sequenced for correct Psl0096 scFv insert, this vector is called Bs3-GLO.

Exemplary experiments showing the functionality of the various bispecific antibodies provided herein, including opsonophagocytic killing (OPK), inhibition of attachment of P. aeruginosa to cells, cytotoxicity, and in vivo vaccination/challenge studies, can be found in PCT Application No. PCT/US2013/068609.

The nucleotide and amino acid sequences of Bs2-GLO, Bs3-GLO and Bs4-GLO are shown below:

Bs2-V2L2MD/Psl0096
Bs2-GLO: Bs2-V2L2MD/Psl0096 Nucleotide-HC
(SEQ ID NO: 10)
CAGGTGCAGCTGCAGGAATCTGGCCCTGGCCTCGTGAAGCCCTCCGAGAC
ACTGTCTCTGACCTGCACCGTGTCCGGCGGCTCCATCTCCCCTTACTACT
GGACCTGGATCAGACAGCCCCCTGGCAAGTGCCTGGAACTGATCGGCTAC
ATCCACTCCTCCGGCTACACCGACTACAACCCCAGCCTGAAGTCCAGAGT
GACCATCTCCGGCGACACCTCCAAGAAGCAGTTCTCCCTGAAGCTGTCCT
CCGTGACCGCCGCTGATACCGCCGTGTACTACTGCGCCAGAGCCGACTGG
GACAGACTGAGAGCCCTGGACATCTGGGGCCAGGGCACAATGGTCACCGT
GTCTAGCGGAGGCGGAGGATCTGGTGGTGGTGGATCTGGCGGCGGAGGAA
GTGGTGGCGGAGGCTCTGATATCCAGCTGACCCAGTCCCCCTCCAGCCTG
TCTGCTTCTGTGGGCGACCGCGTGACCATCACCTGTAGAGCCTCCCAGTC
CATCCGGTCCCACCTGAACTGGTATCAGCAGAAGCCCGGCAAGGCCCCCA
AGCTGCTGATCTACGGCGCCTCCAATCTGCAGTCCGGCGTGCCCTCTAGA
TTCTCCGGATCTGGCTCCGGCACCGACTTTACCCTGACCATCAGCTCCCT
GCAGCCCGAGGACTTCGCCACCTACTACTGCCAGCAGTCTACCGGCGCCT
GGAATTGGTTCGGCTGCGGCACCAAGGTGGAAATCAAGGGCGGAGGGGGA
TCCGGCGGAGGGGGCTCTGAGGTGCAGCTGTTGGAGTCTGGGGGAGGCTT
GGTACAGCCTGGGGGGTCCCTGAGACTCTCCTGTGCAGCCTCTGGATTCA
CCTTTAGCAGCTATGCCATGAACTGGGTCCGCCAGGCTCCAGGGAAGGGG
CTGGAGTGGGTCTCAGCTATTACTATGAGTGGTATTACCGCATACTACAC
CGACGACGTGAAGGGCCGGTTCACCATCTCCAGAGACAATTCCAAGAACA
CGCTATATCTGCAAATGAACAGCCTGAGGGCCGAGGACACGGCCGTATAT
TACTGTGCGAAGGAAGAATTTTTACCTGGAACGCACTACTACTACGGTAT
GGACGTCTGGGGCCAAGGGACCACGGTCACCGTCTCCTCAGCgTCgACCA
AGGGCCCATCcGTCTTCCCCCTGGCACCCTCCTCCAAGAGCACCTCTGGG
GGCACAGCGGCCCTGGGCTGCCTGGTCAAGGACTACTTCCCCGAACCGGT
GACGGTGTCcTGGAACTCAGGCGCtCTGACCAGCGGCGTGCACACCTTCC
CGGCTGTCCTACAGTCCTCAGGACTCTACTCCCTCAGCAGCGTGGTGACC
GTGCCCTCCAGCAGCTTGGGCACCCAGACCTACATCTGCAACGTGAATCA
CAAGCCCAGCAACACCAAGGTGGACAAGAGAGTTGAGCCCAAATCTTGTG
ACAAAACTCACACATGCCCACCGTGCCCAGCACCTGAACTCCTGGGGGGA
CCGTCAGTCTTCCTCTTCCCCCCAAAACCCAAGGACACCCTCATGATCTC
CCGGACCCCTGAGGTCACATGCGTGGTGGTGGACGTGAGCCACGAAGACC
CTGAGGTCAAGTTCAACTGGTACGTGGACGGCGTGGAGGTGCATAATGCC
AAGACAAAGCCGCGGGAGGAGCAGTACAACAGCACGTACCGTGTGGTCAG
CGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCC
AAAGCCAAAGGGCAGCCCCGAGAACCACAGGTcTACACCCTGCCCCCATC
CCGGGAGGAGATGACCAAGAACCAGGTCAGCCTGACCTGCCTGGTCAAAG
GCTTCTATCCCAGCGACATCGCCGTGGAGTGGGAGAGCAATGGGCAGCCG
GAGAACAACTACAAGACCACGCCTCCCGTGCTGGACTCCGACGGCTCCTT
CTTCCTCTATAGCAAGCTCACCGTGGACAAGAGCAGGTGGCAGCAGGGGA
ACGTCTTCTCATGCTCCGTGATGCATGAGGCTCTGCACAACCACTACACG
CAGAAGAGCttaagCCTGTCTCCGGGTAAA
Bs2-GLO: Bs2-V2L2MD/Psl0096 Nucleotide-LC:
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
Bs2-GLO: Bs2-V2L2MD/Psl0096 Amino acid-HC:
(SEQ ID NO: 6)
QVQLQESGPGLVKPSETLSLTCTVSGGSISPYYWTWIRQPPGKCLELIGY
IHSSGYTDYNPSLKSRVTISGDTSKKQFSLKLSSVTAADTAVYYCARADW
DRLRALDIWGQGTMVTVSSGGGGSGGGGSGGGGSGGGGSDIQLTQSPSSL
SASVGDRVTITCRASQSIRSHLNWYQQKPGKAPKLLIYGASNLQSGVPSR
FSGSGSGTDFTLTISSLQPEDFATYYCQQSTGAWNWFGCGTKVEIKGGGG
SGGGGSEVQLLESGGGLVQPGGSLRLSCAASGFTFSSYAMNWVRQAPGKG
LEWVSAITMSGITAYYTDDVKGRFTISRDNSKNTLYLQMNSLRAEDTAVY
YCAKEEFLPGTHYYYGMDVWGQGTTVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYT
QKSLSLSPGK
Bs2-GLO: Bs2-V2L2MD/Psl0096 Amino acid-LC:
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Bs3-V2L2MD/Psl0096
Bs3-GLO: Bs3-V2L2MD/Psl0096 Nucleotide-HC
(SEQ ID NO: 12)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAACTGGGTCCGCCAGGCTCCAGGGAAGGGGCTGGAGTGGGTCTCAGCT
ATTACTATGAGTGGTATTACCGCATACTACACCGACGACGTGAAGGGCCG
GTTCACCATCTCCAGAGACAATTCCAAGAACACGCTATATCTGCAAATGA
ACAGCCTGAGGGCCGAGGACACGGCCGTATATTACTGTGCGAAGGAAGAA
TTTTTACCTGGAACGCACTACTACTACGGTATGGACGTCTGGGGCCAAGG
GACCACGGTCACCGTCTCCTCAGCgTCgACCAAGGGCCCATCcGTCTTCC
CCCTGGCACCCTCCTCCAAGAGCACCTCTGGGGGCACAGCGGCCCTGGGC
TGCCTGGTCAAGGACTACTTCCCCGAACCGGTGACGGTGTCcTGGAACTC
AGGCGCtCTGACCAGCGGCGTGCACACCTTCCCGGCTGTCCTACAGTCCT
CAGGACTCTACTCCCTCAGCAGCGTGGTGACCGTGCCCTCCAGCAGCTTG
GGCACCCAGACCTACATCTGCAACGTGAATCACAAGCCCAGCAACACCAA
GGTGGACAAGAGAGTTGAGCCCAAATCTTGTGACAAAACTCACACATGCC
CACCGTGCCCAGCACCTGAACTCCTGGGGGGACCGTCAGTCTTCCTCTTC
CCCCCAAAACCCAAGGACACCCTCATGATCTCCCGGACCCCTGAGGTCAC
ATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAG
GAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAG
CCCTCCCAGCCCCCATCGAGAAAACCATCTCCAAAGCCAAAGGGCAGCCC
CGAGAACCACAGGTcTACACCCTGCCCCCATCCCGGGAGGAGATGACCAA
GAACCAGGTCAGCCTGACCTGCCTGGTCAAAGGCTTCTATCCCAGCGACA
TCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCT
CACCGTGGACAAGAGCAGGTGGCAGCAGGGGAACGTCTTCTCATGCTCCG
TGATGCATGAGGCTCTGCACAACCACTACACGCAGAAGAGCttaagCCTG
TCTCCGGGTAAAGGCGGAGGGGGATCCGGCGGAGGGGGCTCTCAGGTGCA
GCTGCAGGAATCTGGCCCTGGCCTCGTGAAGCCCTCCGAGACACTGTCTC
TGACCTGCACCGTGTCCGGCGGCTCCATCTCCCCTTACTACTGGACCTGG
ATCAGACAGCCCCCTGGCAAGTGCCTGGAACTGATCGGCTACATCCACTC
CTCCGGCTACACCGACTACAACCCCAGCCTGAAGTCCAGAGTGACCATCT
CCGGCGACACCTCCAAGAAGCAGTTCTCCCTGAAGCTGTCCTCCGTGACC
GCCGCTGATACCGCCGTGTACTACTGCGCCAGAGCCGACTGGGACAGACT
GAGAGCCCTGGACATCTGGGGCCAGGGCACAATGGTCACCGTGTCTAGCG
GAGGCGGAGGATCTGGTGGTGGTGGATCTGGCGGCGGAGGAAGTGGTGGC
GGAGGCTCTGATATCCAGCTGACCCAGTCCCCCTCCAGCCTGTCTGCTTC
TGTGGGCGACCGCGTGACCATCACCTGTAGAGCCTCCCAGTCCATCCGGT
CCCACCTGAACTGGTATCAGCAGAAGCCCGGCAAGGCCCCCAAGCTGCTG
ATCTACGGCGCCTCCAATCTGCAGTCCGGCGTGCCCTCTAGATTCTCCGG
ATCTGGCTCCGGCACCGACTTTACCCTGACCATCAGCTCCCTGCAGCCCG
AGGACTTCGCCACCTACTACTGCCAGCAGTCTACCGGCGCCTGGAATTGG
TTCGGCTGCGGCACCAAGGTGGAAATCAAG
Bs3-GLO: Bs3-V2L2MD/Psl0096 Nucleotide-LC
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
Bs3-GLO: Bs3-V2L2MD/Psl0096 Amino acid-HC
(SEQ ID NO: 8)
EVQLLESGGGLVQPGGSLRLSCAASGFTFSSYAMNWVRQAPGKGLEWVSA
ITMSGITAYYTDDVKGRFTISRDNSKNTLYLQMNSLRAEDTAVYYCAKEE
FLPGTHYYYGMDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGKGGGGSGGGGSQVQLQESGPGLVKPSETLSLTCTVSGGSISPYYWTW
IRQPPGKCLELIGYIHSSGYTDYNPSLKSRVTISGDTSKKQFSLKLSSVT
AADTAVYYCARADWDRLRALDIWGQGTMVTVSSGGGGSGGGGSGGGGSGG
GGSDIQLTQSPSSLSASVGDRVTITCRASQSIRSHLNWYQQKPGKAPKLL
IYGASNLQSGVPSRFSGSGSGTDFTLTISSLQPEDFATYYCQQSTGAWNW
FGCGTKVEIK
Bs3-GLO: Bs3-V2L2MD/Psl0096 Amino acid-LC
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Bs4-V2L2MD/Psl0096
Bs4-GLO: Bs4-V2L2MD/Psl0096 Nucleotide-HC
(SEQ ID NO: 13)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAACTGGGTCCGCCAGGCTCCAGGGAAGGGGCTGGAGTGGGTCTCAGCT
ATTACTATGAGTGGTATTACCGCATACTACACCGACGACGTGAAGGGCCG
GTTCACCATCTCCAGAGACAATTCCAAGAACACGCTATATCTGCAAATGA
ACAGCCTGAGGGCCGAGGACACGGCCGTATATTACTGTGCGAAGGAAGAA
TTTTTACCTGGAACGCACTACTACTACGGTATGGACGTCTGGGGCCAAGG
GACCACGGTCACCGTCTCCTCAGCgTCgACCAAGGGCCCATCcGTCTTCC
CCCTGGCACCCTCCTCCAAGAGCACCTCTGGGGGCACAGCGGCCCTGGGC
TGCCTGGTCAAGGACTACTTCCCCGAACCGGTGACGGTGTCcTGGAACTC
AGGCGCtCTGACCAGCGGCGTGCACACCTTCCCGGCTGTCCTACAGTCCT
CAGGACTCTACTCCCTCAGCAGCGTGGTGACCGTGCCCTCCAGCAGCTTG
GGCACCCAGACCTACATCTGCAACGTGAATCACAAGCCCAGCAACACCAA
GGTGGACAAGAGAGTTGAGCCCAAATCTTGTGGCGGAGGGGGCTCTGGCG
GAGGGggatccCAGGTGCAGCTGCAGGAATCTGGCCCTGGCCTCGTGAAG
CCCTCCGAGACACTGTCTCTGACCTGCACCGTGTCCGGCGGCTCCATCTC
CCCTTACTACTGGACCTGGATCAGACAGCCCCCTGGCAAGTGCCTGGAAC
TGATCGGCTACATCCACTCCTCCGGCTACACCGACTACAACCCCAGCCTG
AAGTCCAGAGTGACCATCTCCGGCGACACCTCCAAGAAGCAGTTCTCCCT
GAAGCTGTCCTCCGTGACCGCCGCTGATACCGCCGTGTACTACTGCGCCA
GAGCCGACTGGGACAGACTGAGAGCCCTGGACATCTGGGGCCAGGGCACA
ATGGTCACCGTGTCTAGCGGAGGCGGAGGATCTGGTGGTGGTGGATCTGG
CGGCGGAGGAAGTGGTGGCGGAGGCTCTGATATCCAGCTGACCCAGTCCC
CCTCCAGCCTGTCTGCTTCTGTGGGCGACCGCGTGACCATCACCTGTAGA
GCCTCCCAGTCCATCCGGTCCCACCTGAACTGGTATCAGCAGAAGCCCGG
CAAGGCCCCCAAGCTGCTGATCTACGGCGCCTCCAATCTGCAGTCCGGCG
TGCCCTCTAGATTCTCCGGATCTGGCTCCGGCACCGACTTTACCCTGACC
ATCAGCTCCCTGCAGCCCGAGGACTTCGCCACCTACTACTGCCAGCAGTC
TACCGGCGCCTGGAATTGGTTCGGCTGCGGCACCAAGGTGGAAATCAAGG
GCGGAGGTGGCTCTGGCGGAGGGggatccGACAAAACTCACACATGCCCA
CCGTGCCCAGCACCTGAACTCCTGGGGGGACCGTCAGTCTTCCTCTTCCC
CCCAAAACCCAAGGACACCCTCATGATCTCCCGGACCCCTGAGGTCACAT
GCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGA
GCAGTACAACAGCACGTACCGTGTGGTCAGCGTCCTCACCGTCCTGCACC
AGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCG
AGAACCACAGGTcTACACCCTGCCCCCATCCCGGGAGGAGATGACCAAGA
ACCAGGTCAGCCTGACCTGCCTGGTCAAAGGCTTCTATCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTATAGCAAGCTCA
CCGTGGACAAGAGCAGGTGGCAGCAGGGGAACGTCTTCTCATGCTCCGTG
ATGCATGAGGCTCTGCACAACCACTACACGCAGAAGAGCttaagCCTGTC
TCCGGGTAAA
Bs4-GLO: Bs4-V2L2MD/Psl0096 Nucleotide-LC
(SEQ ID NO: 11)
GCCATCCAGATGACCCAGTCTCCATCCTCCCTGTCTGCATCTGTAGGAGA
CAGAGTCACCATCACTTGCCGGGCAAGTCAGGGCATTAGAAATGATTTAG
GCTGGTATCAACAGAAGCCAGGGAAAGCCCCTAAACTCCTGATCTATTCT
GCATCCACTTTACAAAGTGGGGTCCCATCAAGGTTCAGCGGCAGTGGATC
TGGCACAGATTTCACTCTCACCATCAGCAGCCTGCAGCCTGAGGATTTTG
CAACTTATTACTGTCTACAAGATTACAATTACCCGTGGACGTTCGGCCAA
GGGACCAAGGTTGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
Bs4-GLO: Bs4-V2L2MD/Psl0096 Amino acid-HC
(SEQ ID NO: 9)
EVQLLESGGGLVQPGGSLRLSCAASGFTFSSYAMNWVRQAPGKGLEWVSA
ITMSGITAYYTDDVKGRFTISRDNSKNTLYLQMNSLRAEDTAVYYCAKEE
FLPGTHYYYGMDVWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCGGGGSGGGGSQVQLQESGPGLVK
PSETLSLTCTVSGGSISPYYWTWIRQPPGKCLELIGYIHSSGYTDYNPSL
KSRVTISGDTSKKQFSLKLSSVTAADTAVYYCARADWDRLRALDIWGQGT
MVTVSSGGGGSGGGGSGGGGSGGGGSDIQLTQSPSSLSASVGDRVTITCR
ASQSIRSHLNWYQQKPGKAPKLLIYGASNLQSGVPSRFSGSGSGTDFTLT
ISSLQPEDFATYYCQQSTGAWNWFGCGTKVEIKGGGGSGGGGSDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
Bs4-GLO: Bs4-V2L2MD/Psl0096 Amino acid-LC
(SEQ ID NO: 7)
AIQMTQSPSSLSASVGDRVTITCRASQGIRNDLGWYQQKPGKAPKLLIYS
ASTLQSGVPSRFSGSGSGTDFTLTISSLQPEDFATYYCLQDYNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Example 2: Bispecific Antibodies Block Attachment of P. aeruginosa to Cultured Epithelial Cells

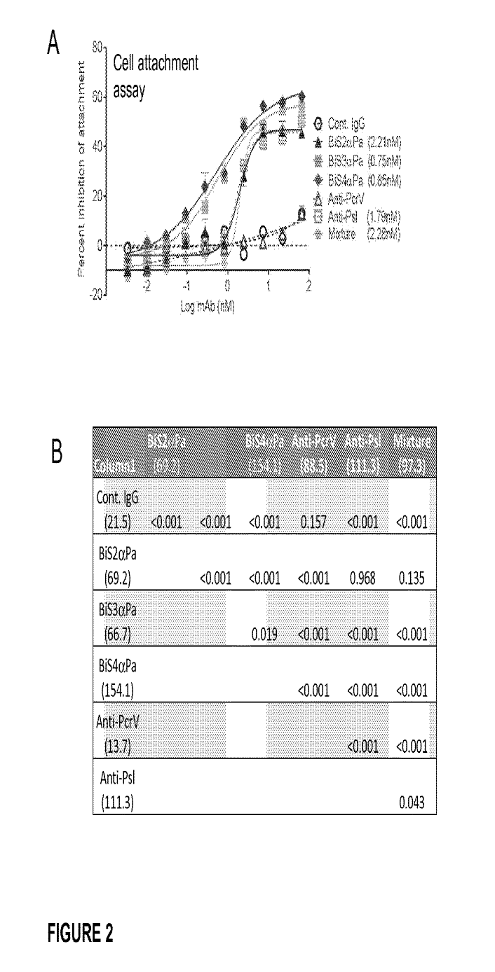

This example shows that Bs2-GLO, Bs3-GLO and Bs4-GLO blocked P. aeruginosa association with epithelial cells. The bispecific antibodies described in Example 1, as well as various control antibodies, were added to a confluent monolayer of A549 cells (an adenocarcinoma human alveolar basal epithelial cell line) grown in opaque 96-well plates (Nunc Nunclon Delta). Log-phase luminescent P. aeruginosa PAO1 strain (PAO1.lux) was added at an MOI of 10. After incubation of PAO1.lux with A549 cells at 37° C. for 1 hour, the A549 cells were washed, followed by addition of LB+0.5% glucose. Bacteria were quantified following a brief incubation at 37° C. Measurements from wells without A549 cells were used to correct for non-specific binding. FIG. 2A shows that all three bispecific antibodies reduced association of PAO1.lux to A549 cells.

### Example 3: Bispecific Antibodies Promote Lysis of Lung Epithelial Cells

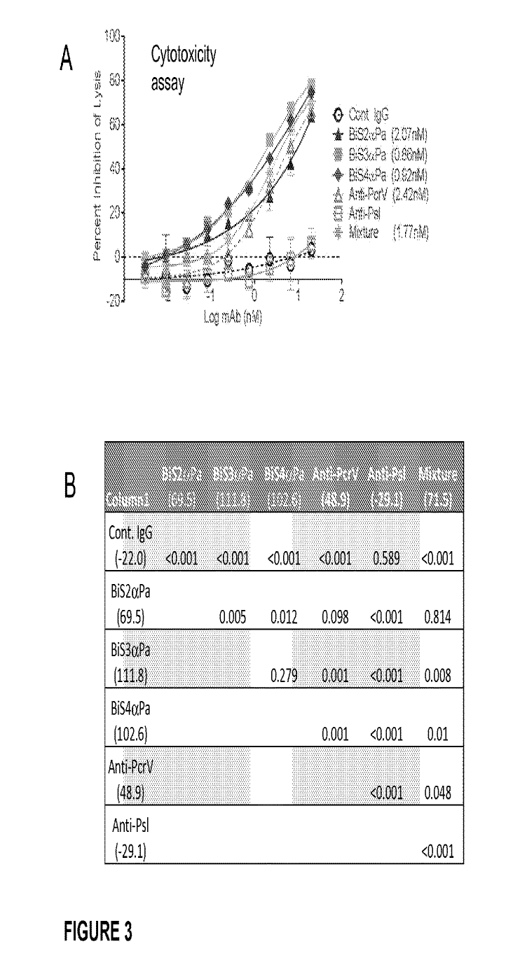

Percent inhibition of cytotoxicity analysis was analyzed for Bs2-GLO, Bs3-GLO, and Bs4-GLO as follows. The antibodies were administered to cultured broncho-epithelial cell line A549 combined with log-phase P. aeruginosa strain 6077 (exoU+) at a MOI of approximately 10. A549 lysis was assayed by measuring release of lactate dehydrogenase (LDH) in the presence of Mabs (including a non-P. aeruginosa reactive IgG control) compared to wells without mAb to determine percent inhibition. All three bispecific antibodies inhibited cell lysis, as shown in FIG. 3A.

### Example 2: Alternate Construction of WapR-004/V2L2 Bispecific Antibodies

To generate Bs2-V2L2-2C, the W4-RAD scFv was fused to N-terminal of V2L2 VH through (G4S)₂ linker (SEQ ID NO: 1). To generate Bs3-V2L2-2C, W4-RAD scFv was fused to C-terminal of CH3 through (G4S)₂ linker (SEQ ID NO: 1). To generate Bs4-V2L2-2C, the W4-RAD scFv was inserted in hinge region, linked by (G4S)₂ linker (SEQ ID NO: 1) on N-terminal and C-terminal of scFv. To generate Bs2-W4-RAD-2C, the V2L2 scFv was fused to the amino-terminus of W4-RAD VH through a (G4S)₂ linker (SEQ ID NO: 1).

Since the combination of WapR-004+V2L2 provide protection against Pseudomonas challenge, bispecific constructs were generated comprising a WapR-004 scFv (W4-RAD) and V2L2 IgG (FIG. 1B). To generate Bs2-V2L2-2C, the W4-RAD scFv is fused to N-terminal of V2L2 VH through (G4S)2 linker (SEQ ID NO: 1). To generate Bs3-V2L2-2C, W4-RAD scFv was fused to C-terminal of CH3 through (G4S)2 linker (SEQ ID NO: 1). To generate Bs4-V2L2-2C, the W4-RAD scFv was inserted in hinge region, linked by (G4S)2 linker (SEQ ID NO: 1) on N-terminal and C-terminal of scFv. To generate Bs2-W4-RAD-2C, the V2L2 scFv was fused to the amino-terminus of W4-RAD VH through a (G4S)2 linker (SEQ ID NO: 1).

To generate the W4-RAD scFv for the Bs3 construct, the W4-RAD VH and VL were amplified by PCR. The primers used to amplify the W4-RAD VH were: W4-RAD VH forward primer: includes (G4S)2 linker (SEQ ID NO: 1) and 22 bp of VH N-terminal sequence (GTAAAGGCGG AGGGGGATCC GGCGGAGGGG GCTCTGAGGT GCAGCTGTTG GAGTCGG (SEQ ID NO: 60)); and W4-RAD VH reverse primer: includes part of (G4S)4 linker (SEQ ID NO: 61) and 22 bp of VH C-terminal sequence (GATCCTCCGC CGCCGCTGCC CCCTCCCCCA GAGCCCCCTC CGCCACTCGA GACGGTGACC AGGGTC (SEQ ID NO: 62). Similarly, the W4-RAD VL was amplified by PCR using the primers: W4-RAD VL forward primer: includes part of (G4S)2 linker (SEQ ID NO: 1) and 22 bp of VL N-terminal sequence (AGGGGGCAGC GGCGGCGGAG GATCTGGGGG AGGGGGCAGC GAAATTGTGT TGACACAGTCTC (SEQ ID NO: 63)); and W4-RAD VL reverse primer: includes part of vector sequence and 22 bp of VL C-terminal sequence (CAATGAATTC GCGGCCGCTC ATTTGATCTC CAGCTTGGTC CCAC (SEQ ID NO: 64)). The overlapping fragments were then fused together to form the W4-RAD scFv.

W4-RAD scFv Sequence in Bs3 Vector: Underlined Sequences are G4S Linker (SEQ ID NO: 40)

(SEQ ID NO: 41)
GGGGSGGGGSEVQLLESGPGLVKPSETLSLTCNVAGGSISPYYWTWIRQP
PGKCLELIGYIHSSGYTDYNPSLKSRVTISGDTSKKQFSLHVSSVTAADT
AVYFCARADWDLLHALDIWGQGTLVTVSSGGGGSGGGGSGGGGSGGGGSE
IVLTQSPSSLSTSVGDRVTITCRASQSIRSHLNWYQQKPGKAPKLLIYGA
SNLQSGVPSRFSGSGSGTDFTLTISSLQPEDFATYYCQQSYSFPLTFGCG
TKLEIK

After the W4-RAD scFv fragment was amplified, it was then gel purified and ligated into the Bs3 vector which had been digested with BamHI/NotI. The ligation was done using the IN-FUSION® system, followed by transformation in STELLAR™ competent cells. Colonies were sequenced to confirm the correct W4-RAD scFv insert.

To generate the Bs3-V2L2-2C, the IgG portion in the Bs3 vector was replaced with V2L2 IgG. Briefly, the Bs3 vector which contains W4-RAD scFv was digested with BssHII/SalI and the resultant vector band was gel purified. Similarly, the vector containing V2L2 vector was digested with BssHII/SalI and the V2L2 insert was gel purified. The V2L2 insert was then ligated with the Bs3-W4-RAD scFv vector and colonies were sequenced to confirm the correct V2L2 IgG insert.

A similar approach was used to generate Bs2-V2L2-2C.

W4-RAD scFv-V2L2 VH Sequences in Bs2 Vector: Underlined Sequences are G4S Linker (SEQ ID NO: 40)

(SEQ ID NO: 42)
EVQLLESGPGLVKPSETLSLTCNVAGGSISPYYWTWIRQPPGKCLELIGY
IHSSGYTDYNPSLKSRVTISGDTSKKQFSLHVSSVTAADTAVYFCARADW
DLLHALDIWGQGTLVTVSSGGGGSGGGGSGGGGSGGGGSEIVLTQSPSSL
STSVGDRVTITCRASQSIRSHLNWYQQKPGKAPKLLIYGASNLQSGVPSR
FSGSGSGTDFTLTISSLQPEDFATYYCQQSYSFPLTFGCGTKLEIKGGGG
SGGGGSEMQLLESGGGLVQPGGSLRLSCAASGFTFSSYAMNWVRQAPGEG
LEWVSAITISGITAYYTDSVKGRFTISRDNSKNTLYLQMNSLRAGDTAVY
YCAKEEFLPGTHYYYGMDVWGQGTTVTVSS

The following primers were used to amplify W4-RAD scFv. VH (forward primer) and VL (reverse primer): W4-RAD VH forward primer for Bs2 vector which includes some intron, 3′ signal peptide and 22 bp of W4-RAD VH N-terminal sequence (TTCTCTCCACAGGTGTACACTCCGAGGTGCAGCTGTTGGAGTCGG (SEQ ID NO: 65)) and W4-RAD VL reverse primer for Bs2 vector: include (G4S)2 linker (SEQ ID NO: 1) and 32 bp of VL C-terminal sequence (CCCCCTCCGC CGGATCCCCC TCCGCCTTTG ATCTCCAGCT TGGTCCCACA GCCGAAAG (SEQ ID NO: 43))

To amplify the V2L2 VH region the following primers were used: V2L2 VH forward primer: includes (G4S)2 linker (SEQ ID NO: 1) and 22 bp of V2L2 VH N-terminal sequence (GGCGGAGGGG GATCCGGCGG AGGGGGCTCT GAGATGCAGC TGTTGGAGTC TGG (SEQ ID NO: 66)), and V2L2 VH reverse primer: includes some of CH1 N-terminal sequence and 22 bp of V2L2 VH C-terminal sequence (ATGGGCCCTT GGTCGACGCT GAGGAGACGG TGACCGTGGTC (SEQ ID NO: 67)).

These primers were then used to amplify V2L2 VH, which was then joined by overlap with W4-RAD scFv and V2L2 VH to get W4-RAD scFv-V2L2-VH. The W4-RAD scFv-V2L2 VH was then ligated into Bs2 vector by gel purifying W4-RAD scFv-V2L2 VH (from overlap PCR); digesting Bs2 vector with BsrGI/SalI, and gel purifying vector band. The W4-RAD scFv-V2L2-VH was then ligated with Bs2 vector by IN-FUSION® system and transformed into STELLAR™ competent cells and the colonies were confirmed for the correct W4-RAD scFv-V2L2 VH insert. To replace VL in Bs2 vector with V2L2 VL, the Bs2 vector which contains W4-RAD scFv-V2L2-VH was digested with BssHII/BsiWI and the vector band was gel purified. The pOE-V2L2 vector was then digested with BssHII/BsiWI and the V2L2 VL insert was gel purified. The V2L2 VL insert was then ligated with Bs2-W4-RAD scFv-V2L2-VH vector and the colonies were sequenced for correct V2L2 IgG insert.

Finally, a similar PCR-based approach was used to generate the Bs4-V2L2-2C construct. The hinge region with linker sequence is shown below:

Hinge Region with Linker Sequence:

-N-terminus of scFv

(SEQ ID NO: 44)

[symbol]

EPKSC [symbol]
CH1 hinge linker
C-terminus of scFv-

(SEQ ID NO: 45)

[symbol]

DKTHTCPPCP [symbol]
linker hinge CH2

W4-RAD scFv sequences in BS4 vector: W4-RAD scFv is in bolded italics with the G4S linkers (SEQ ID NO: 40) underlined in bolded italics; hinge regions are doubled underlined

(SEQ ID NO: 46)

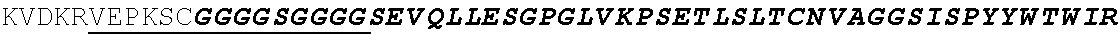

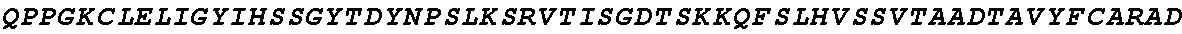

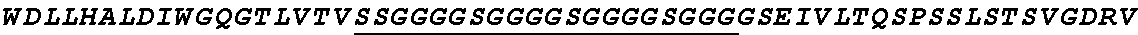

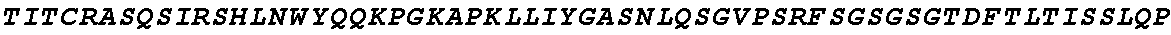

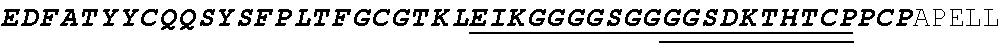

W4-RAD scFv is presented in bolded italics with the G4S linkers (SEQ ID NO:40) underlined in bolded italics

(SEQ ID NO: 68)

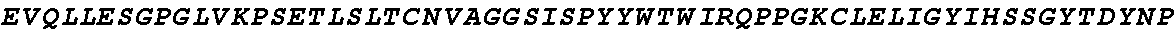

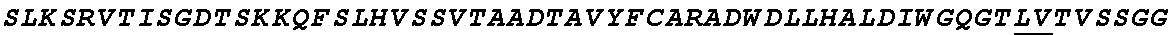

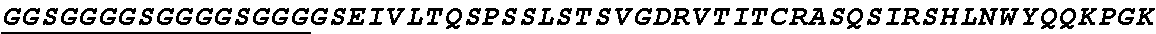

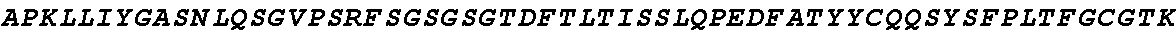

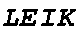

W4-RAD scFv was generated using PCR and the following primers: W4-RAD VH forward primer for Bs4 vector: includes some of linker sequences and 24 bp of W4-RAD VH N-terminal sequence (GAGGTGCAGCTGTTGGAGTCGGGC (SEQ ID NO: 69)); and W4-RAD VL reverse primer for Bs4 vector: includes some hinge sequence, linker and 21 bp of W4-RAD VL C-terminal sequence (GTGTGAGTTT TGTCggatcc CCCTCCGCCA GAGCCACCTC CGCCTTTGAT CTCCAGCTTG GTCCC (SEQ ID NO: 52)).

W4-RAD scFv was then ligated into Bs4 vector to get Bs4-V2L2-2C by gel purifying W4-RAD scFv (from PCR); the Bs4-V2L2 vector was digested with BamHI and the vector band was gel purified. The W4-RAD scFv was ligated with Bs4 vector by IN-FUSION® system and the vector transform STELLAR™ competent cells. Colonies were sequenced for the correct W4-RAD scFv insert.

### Example 3: Alternate Construction of the BS4-GLO Bispecific Antibody

The BS4-GLO (Germlined Lead Optimized) bispecific construct was generated comprising anti-Psl scFv (Psl0096 scfv) and V2L2-MD (VH+VL). The BS4-GLO light chain comprises germlined lead optimized anti-PcrV antibody light chain variable region (i.e., V2L2-MD). The BS4-GLO heavy chain comprises the formula VH-CH1-H1-L1-S-L2-H2-CH2-CH3, wherein CH1 is a heavy chain constant region domain-1, H1 is a first heavy chain hinge region fragment, L1 is a first linker, S is an anti-PcrV ScFv molecule, L2 is a second linker, H2 is a second heavy chain hinge region fragment, CH2 is a heavy chain constant region domain-2, and CH3 is a heavy chain constant region domain-3.

Bs4-GLO light chain:
(SEQ ID NO: 7)
AIQMTQSPSSLSASVGDRVTITCRASQGIRNDLGWYQQKPGKAPKLLIYSASTLQS
GVPSRFSGSGSGTDFTLTISSLQPEDFATYYCLQDYNYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
GLO (germlined lead optimized) V2L2 (i.e., V2L2-MD) light chain variable region is
underlined
Bs4-GLO heavy chain:
(SEQ ID NO: 70)
EMQLLESGGGLVQPGGSLRLSCAASGFTFSSYAMNWVRQAPGEGLEWVSAITIS
GITAYYTDSVKGRFTISRDNSKNTLYLQMNSLRAGDTAVYYCAKEEFLPGTHYY
YGMDVWGQGTTVTVSS[ASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK

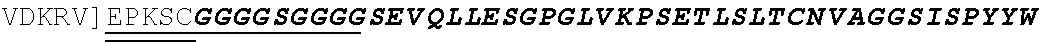

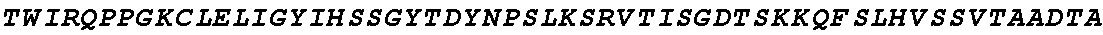

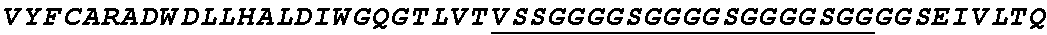

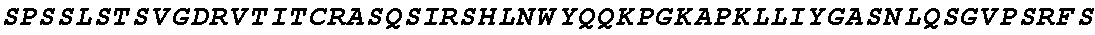

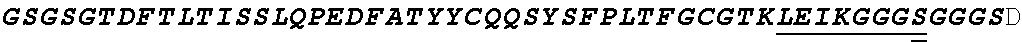

KTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGK
GLO (germlined -lead optimized) V2L2 (i.e., V2L2-MD) heavy chain variable region is underlined; CH1 is bracketed []; GLO (germlined-lead optimized) W4- RAD (i.e., Ps10096) scFv is in bolded italics with the G4S linkers (SEQ ID NO: 40) underlined in bolded italics; hinge regions are doubled underlined.

The disclosure is not to be limited in scope by the specific embodiments described which are intended as single illustrations of individual aspects of the disclosure, and any compositions or methods which are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference. In addition, U.S. Provisional Application Nos. 61/556,645 filed Nov. 7, 2011; 61/624,651 filed Apr. 16, 2012; 61/625,299 filed Apr. 17, 2012; 61/697,585 filed Sep. 6, 2012 and International Application No: PCT/US2012/63639, filed Nov. 6, 2012 (attorney docket no. AEMS-115WO1, entitled “MULTISPECIFIC AND MULTIVALENT BINDING PROTEINS AND USES THEREOF”) are incorporated by reference in their entirety for all purposes.

1. A bispecific antibody comprising a binding domain that binds to Pseudomonas Psl and a binding domain that binds to Pseudomonas PcrV.

2. The bispecific antibody of claim 1, wherein the Psl binding domain comprises a scFv fragment and the PcrV binding domain comprises an intact immunoglobulin.

3. The bispecific antibody of claim 1, wherein the Psl binding domain comprises an intact immunoglobulin and the PcrV binding domain comprises a scFv fragment.

4. The bispecific antibody of claim 2 comprising a Bs-2 molecule, wherein the scFv is fused to the amino-terminus of the VH region of the intact immunoglobulin.

5. The bispecific antibody of claim 2 comprising a Bs-3 molecule, wherein the scFv is fused to the carboxy-terminus of the CH3 region of the intact immunoglobulin.

6. (canceled)

7. (canceled)

8. The bispecific antibody of claim 1, wherein the anti-Psl binding domain comprises a VH and VL region at least 90% identical to the corresponding region of WapR-004-RAD.

9. The bispecific antibody of claim 8, wherein the WapR-004-RAD VH and VL are arranged as a ScFv.

10. (canceled)

11. (canceled)

12. The bispecific antibody of claim 1, wherein the anti-PcrV binding domain comprises VH and VL regions at least 90% identical to the corresponding regions of V2L2.

13. The bispecific antibody of claim 8, comprising Bs-2-GLO, Bs-3-GLO, or Bs-4-GLO.

14. A cell comprising or producing the bispecific antibody of claim 1.

15. An isolated polynucleotide molecule comprising a polynucleotide that encodes the bispecific antibody of claim 1.

16. A vector comprising the polynucleotide of claim 15.

17. A cell comprising the vector of claim 16.

18. A composition comprising the bispecific antibody of claim 1 and a pharmaceutically acceptable carrier.

19. The bispecific antibody of claim 1, which is conjugated to an agent selected from the group consisting of antimicrobial agent, a therapeutic agent, a prodrug, a peptide, a protein, an enzyme, a lipid, a biological response modifier, pharmaceutical agent, a lymphokine, a heterologous antibody or fragment thereof, a detectable label, polyethylene glycol (PEG), and a combination of two or more of any said agents.

20. The bispecific antibody of claim 19, wherein the detectable label is selected from the group consisting of an enzyme, a fluorescent label, a chemiluminescent label, a bioluminescent label, a radioactive label, or a combination of two or more of any said detectable labels.

21. The composition of claim 18, further comprising an antibiotic.

22. The composition of claim 21, wherein the antibiotic is selected from the group consisting of Ciprofloxacin, Meropenem, and a combination thereof.

23. A method of preventing or treating a Pseudomonas infection in a subject in need thereof, comprising administering to a subject an effective amount of a bispecific antibody comprising a binding domain that binds to Pseudomonas Psl and a binding domain that binds to Pseudomonas PcrV, wherein the administration provides a therapeutic effect in the prevention or treatment of the Pseudomonas infection in the subject.

24. The method of claim 23, wherein said bispecific antibody is administered for two or more prevention/treatment cycles.

25. The method of claim 23, wherein the Pseudomonas infection is a P. aeruginosa infection.

26. The method of claim 23, wherein the subject is a human.

27. (canceled)

28. (canceled)
